SEQUENCE LISTING (56 sequences)

SEQ ID NO: 1 - DNA, 3150 bp, Arabidopsis thaliana
  source 1..3150: mol_type = other DNA
atggaaccaaacaccatggccagcttcgacgatgagcatcgtcattcctcattctcggctaagatttgcaaagtctgtggcgatgaggtcaaagacgatgacaatggtcagacttttgtggcgtgtcacgtgtgcgtttacccggtttgcaaaccttgctatgaatatgagcgtagcaacggtaacaaatgttgccctcaatgcaacactctttacaaacgccacaaaggctctccaaaaatcgccggagatgaagaaaacaatggtcctgatgattcagatgatgagttgaatatcaaatatcgccaggatggttcctccattcaccagaattttgcctatggatcggaaaatggagactacaacagtaagcagcaatggcgtccaaatggtcgagccttttcttccaccggaagtgtgttggggaaagactttgaagctgagagagatggctacacagatgcggagtggaaagaacgtgtagataaatggaaagcgaggcaagagaagagaggtttagtgactaaaggagaacaaacaaacgaagacaaagaagatgatgaagaagaatacctagatgctgaagcaagacaacctctctggagaaaagtcccaatctcttcgagcaaaatcagtccttaccgcatcgtgatcgttcttcgccttgtgatcttagttttcttcttccgtttccgtatcttgacaccagccaaagacgcataccctctctggcttatctcagtcatctgcgagatctggttcgctctctcttggatccttgatcagttcccgaaatggtttccgattaaccgagaaacctaccttgaccgtctctctatgagatttgaacgggatggggaaaagaacaagcttgcaccggttgatgtgtttgtcagtacagtggatccgctcaaggaacctcccataatcacagccaacacaattctttcgatcttagccgttgattacccagtgaataaggtgagctgctatgtgtctgatgacggtgcatcaatgctcctgttcgatacattgtccgaaacttccgagtttgcaaggagatgggttccgttttgcaagaagtacaatgttgagccaagagctccagagttttacttctcagagaagattgattacctaaaggataaagtccaaactacattcgtcaaagatcgcagagcaatgaagagagaatatgaagaattcaaagtgaggatcaatgctttagtagctaaagctcaaaagaagcctgaagaaggttgggttatgcaagatggtacaccatggcctgggaataacactcgtgatcatcccgggatgattcaggtgtatctcggcaaagaaggagcattcgacattgatggtaacgaattgccacgtctcgtgtatgtatcaagagagaagcgtcctggttacgcacaccacaagaaagctggagccatgaatgcaatggttcgagtctctgcagtactcactaatgctccattcatgcttaatctcgactgtgaccattacatcaacaacagtaaagccataagagaatcaatgtgctttctaatggatccacagcttggtaaaaagctctgttatgtccaattcccacaaagattcgacggaatcgatcttaacgatcgatacgccaacagaaacatcgtcttcttcgatataaacatgagaggcttagatggaatccaaggaccagtctatgtaggaacaggatgtgtattcaatagaccagcattatacggatacgaacctccggtatcggaaaaacgaaagaaaatgacatgtgattgttggccgtcgtggatctgctgttgttgcggcggaggtaaccgtaatcacaaatcagattcgtcaaagaagaaatcaggaatcaagagcttgttctcgaaactaaagaagaagactaagaagaagagtgatgataaaacgatgagcagctatagtagaaaacgatcatcgacggaggcgatttttgatttggaagatattgaggaagggcttgaaggatatgatgagcttgagaaatcttcgttaatgtcgcagaagaattttgagaagagatttggaatgtctcctgtgtttattgcgtcgacgttgatggagaacggaggattgccggaggcgacgaacacgagctcgttgatcaaagaagctattcatgttattagctgtggttatgaagagaagactgaatggggcaaagagattggatggatttatggatcagtaacagaagatatcctcactggatttagaatgcattgtagaggttggaaatcagtttactgtatgcctaaaagaccagctttcaaaggatcagctccgatcaatttatctgatcggttgcaccaagttcttagatgggctcttggttccgtcgaaatcttctttagtcgtcattgtcctttgtggtatgcttggggaggcaagcttaagattctagaacgtcttgcttacatcaacacaattgtttatcctttcacttctattcctctacttgcctattgtactattccggccgtttgtcttctcaccggcaagttcatcattcctacgatcaacaatttcgcaagcatatggttcttagctctgttcctatcaatcattgcaacggctatcttagaactccggtggagtggtgtaagcatcaatgacctctggcgtaacgagcaattctgggtgattggcggtgtctcagctcatctcttcgctgtctttcaaggtcttcttaaagttctctttggtgttgacactaacttcaccgtcacgtcaaaaggagccagtgacgaagccgatgagtttggagatctttacctctttaaatggacaacccttctcatccctccgacaactctcattattctcaacatggtcggtgttgttgccggagtttctgacgctattaacaatggatatggttcttggggaccgctttttgggaaactgtttttcgcgttttgggtcattgttcatctttatcctttcctcaaaggtttgatgggtagacaaaaccgaacaccgacgattgttgttctttggtctatccttctcgcttcgatcttctctttggtttgggttcggattgatccgttcttgccgaaacagactggtccgttactgaagcaatgtggcgtcgactgttaa

SEQ ID NO: 2 - protein, 1049 aa, Arabidopsis thaliana
MEPNTMASFDDEHRHSSFSAKICKVCGDEVKDDDNGQTFVACHVCVYPVCKPCYEYERSNGNKCCPQCNTLYKRHKGSPKIAGDEENNGPDDSDDELNIKYRQDGSSIHQNFAYGSENGDYNSKQQWRPNGRAFSSTFSVLGKDFEAERDGYTDAEWKERVDKWKARQEKRGLVTKGEQTNEDKEDDEEEYLDAEARQPLWRKVPISSSKISPYRIVIVLRLVILVFFFRFRILTPAKDAYPLWLISVICEIWFALSWILDQFPKWFPINRETYLDRLSMRFERDGEKNKLAPVDVFVSTVDPLKEPPIITANTILSILAVDYPVNKVSCYVSDDGASMLLFDTLSETSEFARRWVPFCKKYNVEPRAPEFYFSEKIDYLKDKVQTTFVKDRRAMKREYEEFKVRINALVAKAQKKPEEGWVMQDGTPWPGNNTRDHPGMIQVYLGKEGAFDIDGNELPRLVYVSREKRPGYAHHKKAGAMNAMVRVSAVLTNAPFMLNLDCDHYINNSKAIRESMCFLMDPQLGKKLCYVQFPQRFDGIDLNDRYANRNIVFFDINMRGLDGIQGPVYVGTGCVFNRPALYGYEPPVSEKRKKMTCDCWPSWICCCCGGGNRNHKSDSSKKKSGIKSLFSKLKKKTKKKSDDKTMSSYSRKRSSTEAIFDLEDIEEGLEGYDELEKSSLMSQKNFEKRFGMSPVFIASTLMENGGLPEATNTSSLIKEAIHVISCGYEEKTEWGKEIGWIYGSVTEDILTGFRMHCRGWKSVYCMPKRPAFKGSAPINLSDRLHQVLRWALGSVEIFFSRHCPLWYAWGGKLKILERLAYINTIVYPFTSIPLLAYCTIPAVCLLTGKFIIPTINNFASIWFLALFLSIIATAILELRWSGVSINDLWRNEEFWVIGGVSAHLFAVFQGLLKVLFGVDTNFTVTSKGASDEADEFGDLYLFKWTTLLIPPTTLIILAMVGVVAGVSDAINNGYGSWGPLFGKLFFAFWVIVHLYPFLKGLMGRQNRTPTIVVLWSILLASIFSLVWVRIDPFLPKQTGPLLKQCGVDC

SEQ ID NO: 3 - DNA, 3150 bp, synthetic construct
  source 1..3150: mol_type = other DNA
atggaaccaaacaccatggccagcttcgacgatgagcatcgtcattcctcattctcggctaagatttgcaaagtctgtggcgatgaggtcaaagacgatgacaatggtcagacttttgtggcgtgtcacgtgtgcgtttacccggtttgcaaacctttctatgaatatgagcgtagcaacggtaacaaatgttgccctcaatgcaacactctttacaaacgccacaaaggctctccaaaaatcgccggagatgaagaaaacaatggtcctgatgattcagatgatgagttgaatatcaaatatcgccaggatggttcctccattcaccagaattttgcctatggatcggaaaatggagactacaacagtaagcagcaatggcgtccaaatggtcgagccttttcttccaccggaagtgtgttggggaaagactttgaagctgagagagatggctacacagatgcggagtggaaagaacgtgtagataaatggaaagcgaggcaagagaagagaggtttagtgactaaaggagaacaaacaaacgaagacaaagaagatgatgaagaagaatacctagatgctgaagcaagacaacctctctggagaaaagtcccaatctcttcgagcaaaatcagtccttaccgcatcgtgatcgttcttcgccttgtgatcttagttttcttcttccgtttccgtatcttgacaccagccaaagacgcataccctctctggcttatctcagtcatctgcgagatctggttcgctctctcttggatccttgatcagttcccgaaatggtttccgattaaccgagaaacctaccttgaccgtctctctatgagatttgaacgggatggggaaaagaacaagcttgcaccggttgatgtgtttgtcagtacagtggatccgctcaaggaacctcccataatcacagccaacacaattctttcgatcttagccgttgattacccagtgaataaggtgagctgctatgtgtctgatgacggtgcatcaatgctcctgttcgatacattgtccgaaacttccgagtttgcaaggagatgggttccgttttgcaagaagtacaatgttgagccaagagctccagagttttacttctcagagaagattgattacctaaaggataaagtccaaactacattcgtcaaagatcgcagagcaatgaagagagaatatgaagaattcaaagtgaggatcaatgctttagtagctaaagctcaaaagaagcctgaagaaggttgggttatgcaagatggtacaccatggcctgggaataacactcgtgatcatcccgggatgattcaggtgtatctcggcaaagaaggagcattcgacattgatggtaacgaattgccacgtctcgtgtatgtatcaagagagaagcgtcctggttacgcacaccacaagaaagctggagccatgaatgcaatggttcgagtctctgcagtactcactaatgctccattcatgcttaatctcgactgtgaccattacatcaacaacagtaaagccataagagaatcaatgtgctttctaatggatccacagcttggtaaaaagctctgttatgtccaattcccacaaagattcgacggaatcgatcttaacgatcgatacgccaacagaaacatcgtcttcttcgatataaacatgagaggcttagatggaatccaaggaccagtctatgtaggaacaggatgtgtattcaatagaccagcattatacggatacgaacctccggtatcggaaaaacgaaagaaaatgacatgtgattgttggccgtcgtggatctgctgttgttgcggcggaggtaaccgtaatcacaaatcagattcgtcaaagaagaaatcaggaatcaagagcttgttctcgaaactaaagaagaagactaagaagaagagtgatgataaaacgatgagcagctatagtagaaaacgatcatcgacggaggcgatttttgatttggaagatattgaggaagggcttgaaggatatgatgagcttgagaaatcttcgttaatgtcgcagaagaattttgagaagagatttggaatgtctcctgtgtttattgcgtcgacgttgatggagaacggaggattgccggaggcgacgaacacgagctcgttgatcaaagaagctattcatgttattagctgtggttatgaagagaagactgaatggggcaaagagattggatggatttatggatcagtaacagaagatatcctcactggatttagaatgcattgtagaggttggaaatcagtttactgtatgcctaaaagaccagctttcaaaggatcagctccgatcaatttatctgatcggttgcaccaagttcttagatgggctcttggttccgtcgaaatcttctttagtcgtcattgtcctttgtggtatgcttggggaggcaagcttaagattctagaacgtcttgcttacatcaacacaattgtttatcctttcacttctattcctctacttgcctattgtactattccggccgtttgtcttctcaccggcaagttcatcattcctacgatcaacaatttcgcaagcatatggttcttagctctgttcctatcaatcattgcaacggctatcttagaactccggtggagtggtgtaagcatcaatgacctctggcgtaacgagcaattctgggtgattggcggtgtctcagctcatctcttcgctgtctttcaaggtcttcttaaagttctctttggtgttgacactaacttcaccgtcacgtcaaaaggagccagtgacgaagccgatgagtttggagatctttacctctttaaatggacaacccttctcatccctccgacaactctcattattctcaacatggtcggtgttgttgccggagtttctgacgctattaacaatggatatggttcttggggaccgctttttgggaaactgtttttcgcgttttgggtcattgttcatctttatcctttcctcaaaggtttgatgggtagacaaaaccgaacaccgacgattgttgttctttggtctatccttctcgcttcgatcttctctttggtttgggttcggattgatccgttcttgccgaaacagactggtccgttactgaagcaatgtggcgtcgactgttaa

SEQ ID NO: 4 - protein, 1049 aa, synthetic construct
MEPNTMASFDDEHRHSSFSAKICKVCGDEVKDDDNGQTFVACHVCVYPVCKPFYEYERSNGNKCCPQCNTLYKRHKGSPKIAGDEENNGPDDSDDELNIKYRQDGSSIHQNFAYGSENGDYNSKQQWRPNGRAFSSTGSVLGKDFEAERDGYTDAEWKERVDKWKARQEKRGLVTKGEQTNEDKEDDEEEYLDAEARQPLWRKVPISSSKISPYRIVIVLRLVILVFFFRFRILTPAKDAYPLWLISVICEIWFALSWILDQFPKWFPINRETYLDRLSMRFERDGEKNKLAPVDVFVSTVDPLKEPPIITANTILSILAVDYPVNKVSCYVSDDGASMLLFDTLSETSEFARRWVPFCKKYNVEPRAPEFYFSEKIDYLKDKVQTTFVKDRRAMKREYEEFKVRINALVAKAQKKPEEGWVMQDGTPWPGNNTRDHPGMIQVYLGKEGAFDIDGNELPRLVYVSREKRPGYAHHKKAGAMNAMVRVSAVLTNAPFMLNLDCDHYINNSKAIRESMCFLMDPQLGKKLCYVQFPQRFDGIDLNDRYANRNIVFFDINMRGLDGIQGPVYVGTGCVFNRPALYGYEPPVSEKRKKMTCDCWPSWICCCCGGGNRNHKSDSSKKKSGIKSLFSKLKKKTKKKSDDKTMSSYSRKRSSTEAIFDLEDIEEGLEGYDELEKSSLMSQKNFEKRFGMSPVFIASTLMENGGLPEATNTSSLIKEAIHVISCGYEEKTEWGKEIGWIYGSVTEDILTGFRMHCRGWKSVYCMPKRPAFKGSAPINLSDRLHQVLRWALGSVEIFFSRHCPLWYAWGGKLKILERLAYINTIVYPFTSIPLLAYCTIPAVCLLTGKFIIPTINNFASIWFLALFLSIIATAILELRWSGVSINDLWRNEQFWVIGGVSAHLFAVFQGLLKVLFGVDTNFTVTSKGASDEADEFGDLYLFKWTTLLIPPTTLIILNMVGVVAGVSDAINNGYGSWGPLFGKLFFAFWVIVHLYPFLKGLMGRQNRTPTIVVLWSILLASIFSLVWVRIDPFLPKQTGPLLKQCGVDC

SEQ ID NO: 5 - DNA, 3255 bp, Arabidopsis thaliana
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 6 - protein, 1084 aa, Arabidopsis thaliana
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 7 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgataatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 8 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDNGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 9 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtaatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 10 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCNHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 11 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcaattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 12 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVNCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 13 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattaataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 14 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGINRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 15 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacaatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 16 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHNRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 17 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaaaatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 18 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTENILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 19 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacaaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 20 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNKQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 21 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcgatacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 22 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVDTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 23 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatagttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 24 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYSSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 25 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaagaatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 26 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKESAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 27 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattgaaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 28 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIEGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 29 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgatttttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 30 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVIFGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 31 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgtttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 32 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWAFGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 33 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttctctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 34 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFSQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 35 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatgaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 36 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHEVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 37 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttaatacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 38 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVNTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 39 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatttgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 40 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVFDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 41 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttgcatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 42 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLAWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 43 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttaccgaagatattcttacgggttttaagatgcattctcatggttggagatctgtttattgtacaccaaagttagcggctttcaaaggatcagctccaatcaatctttcggatcgtctccatcaagttcttcgagcggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 44 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVTEDILTGFKMHSHGWRSVYCTPKLAAFKGSAPINLSDRLHQVLRAALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 45 - DNA, 3255 bp, synthetic construct
  source 1..3255: mol_type = other DNA
atgaacaccggtggtcggttaatcgccggttctcacaacaggaatgagtttgtcctcattaatgccgatgagaatgcccgaataagatcagtccaagagctgagtggacagacatgtcaaatctgcagagatgagatcgaattgactgttgatggagaaccgtttgtggcatgtaacgaatgtgcattccctgtgtgtagaccttgctatgagtacgaaagacgagaaggcaatcaagcttgtccacagtgcaaaacccgtttcaaacgtcttaaaggaagtccaagagttgaaggtgatgaagaggaagatgacattgatgatttagacaatgagtttgagtatggaaataatgggattggatttgatcaggtttctgaaggtatgtcaatctctcgtcgcaactccggtttcccacaatctgatttggattcagctccacctggctctcagattccattgctgacttacggcgacgaggacgttgagatttcttctgatagacatgctcttattgttcctccttcacttggtggtcatggcaatagagttcatcctgtttctctttctgacccgaccgtggctgcacatccaaggcctatggtacctcagaaagatcttgcggtttatggttatggaagtgtcgcttggaaagatcggatggaggaatggaagagaaagcagaatgagaaacttcaggttgttaggcatgaaggagatcctgattttgaagatggtgatgatgctgattttccaatgatggatgagggaaggcagccattgtctaggaagataccaatcaaatcgagcaagataaatccttaccggatgttaattgtgctacgtcttgtgattcttggtctcttctttcactaccgtattcttcaccccgtcaaagatgcatatgctttgtggcttatttctgttatatgtgagatatggtttgctgtttcatgggttcttgatcagttccctaaatggtaccctatcgagcgagaaacgtacttggaccgactctcattaagatatgagaaagaagggaaaccgtcgggactatcccctgtggatgtatttgttagtacagtggatccattgaaagagcctccgcttattactgcaaatactgtcttgtctattcttgctgttgattatcctgtcgataaggttgcttgttacgtatctgatgatggtgctgctatgcttactttcgaagctctttctgagaccgctgaattcgcaaggaaatgggttcctttctgcaagaaatattgtattgagcctcgtgctcccgaatggtatttctgccataaaatggactacttgaagaataaagttcatcccgcatttgttagggagcggcgagccatgaagagagattatgaagaattcaaagtaaagatcaatgctttagtagcaacagcacagaaagtgcctgaggatggttggactatgcaagacggtacaccttggcccggtaatagtgtgcgagatcatcctggcatgattcaggtcttccttggaagtgacggtgttcgtgatgtcgaaaacaacgagttgcctcgattagtttacgtttctcgtgagaagagacccggatttgatcaccataagaaggctggagctatgaattccctgatacgagtctctggggttctatcaaatgctccttaccttctgaatgtcgattgtgatcactacatcaacaatagcaaagctcttagagaagcaatgtgtttcatgatggatcctcagtcaggaaagaaaatctgttatgttcagttccctcaaaggttcgatgggattgataggcacgatcgatactcaaatcgcaatgttgtgttctttgatatcaatatgaaaggtttggatgggctacaagggcctatatacgtcggtacaggttgtgttttcaggaggcaagcgctttacggatttgatgcaccgaagaagaagaagggcccacgtaagacatgcaattgctggccaaaatggtgtctcctatgttttggttcaagaaagaatcgtaaagcaaagacagtggctgcggataagaagaagaagaatagggaagcgtcaaagcagatccacgcattagaaaatatcgaagagggccgcgtcactaaaggttctaacgtagaacagtcaaccgaggcaatgcaaatgaagttggagaagaaatttgggcagtctcctgtatttgttgcatctgcgcgtatggagaatggtgggatggctagaaacgcaagcccggcttgtctgcttaaagaagccatccaagtcattagttgcggatatgaagataaaactgaatggggaaaagagattgggtggatctatggttctgttatcgaagatattcttacgggttttaagatgcattctcatggttggagatctgtttattgtacaccaaagttagcggctttcaaaggatcagctccaatcaatctttcggatcgtctccatcaagttcttcgatgggcgcttgggtcggttgagattttcttgagtaggcattgtcctatttggtatggttatggaggtgggttgaaatggcttgagcggttgtcctacattaactctgtggtttacccgtggacctctctaccgctcatcgtttactgttctctccctgccatctgtcttctcactggaaaattcatcgttcccgagattagcaactatgcgagtatcctcttcatggcgctcttctcgtcgattgcaataacgggtattctcgagatgcaatggggcaaagttgggatcgatgattggtggagaaacgaacagttttgggtcattggaggtgtttctgcgcatctgtttgctctcttccaaggtctcctcaaggttcttgctggtgtcgacactaacttcacagtcacatcaaaagcagctgatgatggagagttctctgacctttacctcttcaaatggacttcacttctcatccctccaatgactctactcatcataaacgtcattggagtcatagtcggagtctctgatgccatcagcaatggatacgactcgtggggaccgcttttcggaagactgttctttgcactttgggtcatcattcatctttacccgttccttaaaggtttgcttgggaaacaagatagaatgccaaccattattgtcgtctggtccatcctcctggcctcgattcttacacttctttgggtccgggttaatccgtttgtggcgaaaggcggtcctattctcgagatctgtggtttagactgcttgtga

SEQ ID NO: 46 - protein, 1084 aa, synthetic construct
MNTGGRLIAGSHNRNEFVLINADENARIRSVQELSGQTCQICRDEIELTVDGEPFVACNECAFPVCRPCYEYERREGNQACPQCKTRFKRLKGSPRVEGDEEEDDIDDLDNEFEYGNNGIGFDQVSEGMSISRRNSGFPQSDLDSAPPGSQIPLLTYGDEDVEISSDRHALIVPPSLGGHGNRVHPVSLSDPTVAAHPRPMVPQKDLAVYGYGSVAWKDRMEEWKRKQNEKLQVVRHEGDPDFEDGDDADFPMMDEGRQPLSRKIPIKSSKINPYRMLIVLRLVILGLFFHYRILHPVKDAYALWLISVICEIWFAVSWVLDQFPKWYPIERETYLDRLSLRYEKEGKPSGLSPVDVFVSTVDPLKEPPLITANTVLSILAVDYPVDKVACYVSDDGAAMLTFEALSETAEFARKWVPFCKKYCIEPRAPEWYFCHKMDYLKNKVHPAFVRERRAMKRDYEEFKVKINALVATAQKVPEDGWTMQDGTPWPGNSVRDHPGMIQVFLGSDGVRDVENNELPRLVYVSREKRPGFDHHKKAGAMNSLIRVSGVLSNAPYLLNVDCDHYINNSKALREAMCFMMDPQSGKKICYVQFPQRFDGIDRHDRYSNRNVVFFDINMKGLDGLQGPIYVGTGCVFRRQALYGFDAPKKKKGPRKTCNCWPKWCLLCFGSRKNRKAKTVAADKKKKNREASKQIHALENIEEGRVTKGSNVEQSTEAMQMKLEKKFGQSPVFVASARMENGGMARNASPACLLKEAIQVISCGYEDKTEWGKEIGWIYGSVIEDILTGFKMHSHGWRSVYCTPKLAAFKGSAPINLSDRLHQVLRWALGSVEIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSKAADDGEFSDLYLFKWTSLLIPPMTLLIINVIGVIVGVSDAISNGYDSWGPLFGRLFFALWVIIHLYPFLKGLLGKQDRMPTIIVVWSILLASILTLLWVRVNPFVAKGGPILEICGLDCL

SEQ ID NO: 47 - DNA, 3081 bp, Arabidopsis thaliana
  source 1..3081: mol_type = other DNA
atggaagctagcgccggtcttgtcgccggttctcataaccgtaatgaactagtcgtcattcacaaccatgaagagccaaagcctctgaagaatctagatggacaattctgtgagatatgtggagatcagattggtttaacagtagaaggagacctcttcgtagcttgcaatgagtgtggttttccggcgtgtagaccttgctatgagtacgagagaagagaaggaacacaaaactgtcctcagtgtaagactcgttacaagcgtctcagaggaagcccaagagtggagggagatgaagacgaagaagatattgatgatattgagtatgaatttaatatcgaacatgaacaagataagcataagcattctgctgaggctatgctttatgggaaaatgagctatggaagaggtcctgaggatgatgagaatgggagattcccacctgttatagctggtggtcatagtggagaatttccagttggaggaggttatggtaatggagaacatgggcttcataagcgtgtgcacccatatccatcatctgaagctgggagtgagggaggatggcgggaaagaatggatgactggaagctccagcatggaaatcttgggccagaaccagatgatgatcctgagatgggactgatcgacgaggcacggcagccactctcgcggaaagttcccattgcctcaagcaagatcaatccatatcggatggtcatcgtcgctaggcttgtgattctagcagtttttctgcggtataggctcttgaatccagtgcatgatgctctgggattatggctgacctctgtgatctgtgaaatctggttcgctgtctcttggattcttgatcagttccccaagtggttccctattgaacgtgagacctatctagatcggctttccctcaggtacgagagagaaggtgaaccaaatatgcttgcccctgtagatgtctttgtcagtacggtggacccattgaaggagcctcccctcgtcacatccaacactgtgctgtcaatcttggccatggactacccagttgagaaaatctcctgctatgtctctgacgacggtgcttcaatgcttacattcgaatctctctcggaaactgctgagtttgcaagaaaatgggttcccttctgtaagaaattctccatagagccacgggcaccggagatgtacttcacgttgaaagttgattatctccaggacaaagtccacccaacatttgttaaggaacgtcgagccatgaagagagaatatgaggagttcaaggtcaggatcaacgctcaagtggcgaaggcctcaaaggttcctctagaaggttggatcatgcaagatggaacaccgtggccagggaacaacaccaaggaccaccccggtatgatccaagtcttcctcggccacagcggaggatttgatgtcgaagggcatgagcttcctcggcttgtgtacgtgtcccgtgagaagcgtcctggttttcaacaccacaagaaagctggcgccatgaatgccctggttcgagtggcaggcgtactcacaaatgctcctttcatgctgaacttggactgtgatcactatgtaaacaacagcaaggccgtgagggaagcaatgtgttttttgatggatcctcagattggaaagaaggtctgctatgttcagttccctcaaaggtttgatggcattgacacaaacgatcgttacgccaacagaaacacagtcttctttgatatcaatatgaaaggtctagatggaatccaaggtccagtttacgttggtactggttgtgttttcaaacgacaagctctgtatggttatgaaccaccaaagggtcctaaacgtccaaagatgataagctgtggttgttgtccttgctttgggcgccggagaaagaataagaaattttccaagaatgacatgaatggtgacgtagcagcccttggaggagcagaaggtgataaggagcatttgatgtctgaaatgaactttgagaaaacatttgggcaatcatccatctttgtaacctcaactttgatggaagaaggtggtgttcctccgtcatcaagtcctgcagtgctccttaaagaggcaatccatgtcataagctgcggttatgaagacaagactgagtggggaactgagctgggttggatctatggctctatcacagaggatattttgacgggattcaagatgcattgccgtggatggaggtctatttactgcatgcctaagaggcctgcattcaaaggttcagctcctattaatctatcagacaggttaaaccaggttttgcgttgggcacttggatcggttgagatatttttcagccggcacagtcctctctggtatggctacaaaggaggcaaactcaagtggcttgagcgttttgcttatgccaacacaacaatctaccccttcacatctataccacttcttgcctactgtatccttccagccatctgtctccttactgacaaattcatcatgccaccgataagcacatttgctagtctcttcttcatctcactgtttatgtcgatcattgtaacgggaatcttggaattgagatggagcggagttagcattgaggaatggtggagaaacgagcaattctgggtcattggaggaatctcagctcatctctttgcggttgtccaaggtctcctcaaaatcttagcaggcattgacacaaacttcaccgtcacatcaaaggcaacagatgatgatgactttggagaactttacgcattcaaatggacaacactgctgatccctccaacaactgtcttaatcataaacattgttggcgttgttgcaggcatctcagatgccattaacaatggatatcagtcttggggacctctatttggtaaactcttcttctccttttgggtcattgttcatctctacccattcctcaaaggtctgatgggtagacagaacagaacaccaaccattgtggtgatttggtcagtgttattggcatctatcttctctttgctttgggtaagaattgatccttttgtgctcaagaccaaaggacctgacacttccaagtgtggcatcaactgctga

SEQ ID NO: 48 - protein, 1026 aa, Arabidopsis thaliana
MEASAGLVAGSHNRNELVVIHNHEEPKPLKNLDGQFCEICGDQIGLTVEGDLFVACNECGFPACRPCYEYERREGTQNCPQCKTRYKRLRGSPRVEGDEDEEDIDDIEYEFNIEHEQDKHKHSAEAMLYGKMSYGRGPEDDENGRFPPVIAGGHSGEFPVGGGYGNGEHGLHKRVHPYPSSEAGSEGGWRERMDDWKLQHGNLGPEPDDDPEMGLIDEARQPLSRKVPIASSKINPYRMVIVARLVILAVFLRYRLLNPVHDALGLWLTSVICEIWFAVSWILDQFPKWFPIERETYLDRLSLRYEREGEPNMLAPVDVFVSTVDPLKEPPLVTSNTVLSILAMDYPVEKISCYVSDDGASMLTFESLSETAEFARKWVPFCKKFSIEPRAPEMYFTLKVDYLQDKVHPTFVKERRAMKREYEEFKVRINAQVAKASKVPLEGWIMQDGTPWPGNNTKDHPGMIQVFLGHSGGFDVEGHELPRLVYVSREKRPGFQHHKKAGAMNALVRVAGVLTNAPFMLNLDCDHYVNNSKAVREAMCFLMDPQIGKKVCYVQFPQRFDGIDTNDRYANRNTVFFDINMKGLDGIQGPVYVGTGCVFKRQALYGYEPPKGPKRPKMISCGCCPCFGRRRKNKKFSKNDMNGDVAALGGAEGDKEHLMSEMNFEKTFGQSSIFVTSTLMEEGGVPPSSSPAVLLKEAIHVISCGYEDKTEWGTELGWIYGSITEDILTGFKMHCRGWRSIYCMPKRPAFKGSAPINLSDRLNQVLRWALGSVEIFFSRHSPLWYGYKGGKLKWLERFAYANTTIYPFTSIPLLAYCILPAICLLTDKFIMPPISTFASLFFISLFMSIIVTGILELRWSGVSIEEWWRNEQFWVIGGISAHLFAVVQGLLKILAGIDTNFTVTSKATDDDDFGELYAFKWTTLLIPPTTVLIINIVGVVAGISDAINNGYQSWGPLFGKLFFSFWVIVHLYPFLKGLMGRQNRTPTIVVIWSVLLASIFSLLWVRIDPFVLKTKGPDTSKCGINC

SEQ ID NO: 49 - DNA, 3081 bp, synthetic construct
  source 1..3081: mol_type = other DNA
atggaagctagcgccggtcttgtcgccggttctcataaccgtaatgaactagtcgtcattcacaaccatgaagagccaaagcctctgaagaatctagatggacaattctgtgagatatgtggagatcagattggtttaacagtagaaggagacctcttcgtagcttgcaatgagtgtggttttccggcgtgtagaccttgctatgagtacgagagaagagaaggaacacaaaactgtcctcagtgtaagactcgttacaagcgtctcagaggaagcccaagagtggagggagatgaagacgaagaagatattgatgatattgagtatgaatttaatatcgaacatgaacaagataagcataagcattctgctgaggctatgctttatgggaaaatgagctatggaagaggtcctgaggatgatgagaatgggagattcccacctgttatagctggtggtcatagtggagaatttccagttggaggaggttatggtaatggagaacatgggcttcataagcgtgtgcacccatatccatcatctgaagctgggagtgagggaggatggcgggaaagaatggatgactggaagctccagcatggaaatcttgggccagaaccagatgatgatcctgagatgggactgatcgacgaggcacggcagccactctcgcggaaagttcccattgcctcaagcaagatcaatccatatcggatggtcatcgtcgttaggcttgtgattctagcagtttttctgcggtataggctcttgaatccagtgcatgatgctctgggattatggctgacctctgtgatctgtgaaatctggttcgctgtctcttggattcttgatcagttccccaagtggttccctattgaacgtgagacctatctagatcggctttccctcaggtacgagagagaaggtgaaccaaatatgcttgcccctgtagatgtctttgtcagtacggtggacccattgaaggagcctcccctcgtcacatccaacactgtgctgtcaatcttggccatggactacccagttgagaaaatctcctgctatgtctctgacgacggtgcttcaatgcttacattcgaatctctctcggaaactgctgagtttgcaagaaaatgggttcccttctgtaagaaattctccatagagccacgggcaccggagatgtacttcacgttgaaagttgattatctccaggacaaagtccacccaacatttgttaaggaacgtcgagccatgaagagagaatatgaggagttcaaggtcaggatcaacgctcaagtggcgaaggcctcaaaggttcctctagaaggttggatcatgcaagatggaacaccgtggccagggaacaacaccaaggaccaccccggtatgatccaagtcttcctcggccacagcggaggatttgatgtcgaagggcatgagcttcctcggcttgtgtacgtgtcccgtgagaagcgtcctggttttcaacaccacaagaaagctggcgccatgaatgccctggttcgagtggcaggcgtactcacaaatgctcctttcatgctgaacttggactgtgatcactatgtaaacaacagcaaggccgtgagggaagcaatgtgttttttgatggatcctcagattggaaagaaggtctgctatgttcagttccctcaaaggtttgatggcattgacacaaacgatcgttacgccaacagaaacacagtcttctttgatatcaatatgaaaggtctagatggaatccaaggtccagtttacgttggtactggttgtgttttcaaacgacaagctctgtatggttatgaaccaccaaagggtcctaaacgtccaaagatgataagctgtggttgttgtccttgctttgggcgccggagaaagaataagaaattttccaagaatgacatgaatggtgacgtagcagcccttggaggagcagaaggtgataaggagcatttgatgtctgaaatgaactttgagaaaacatttgggcaatcatccatctttgtaacctcaactttgatggaagaaggtggtgttcctccgtcatcaagtcctgcagtgctccttaaagaggcaatccatgtcataagctgcggttatgaagacaagactgagtggggaactgagctgggttggatctatggctctatcacagaggatattttgacgggattcaagatgcattgccgtggatggaggtctatttactgcatgcctaagaggcctgcattcaaaggttcagctcctattaatctatcagacaggttaaaccaggttttgcgttgggcacttggatcggttgagatatttttcagccggcacagtcctctctggtatggctacaaaggaggcaaactcaagtggcttgagcgttttgcttatgccaacacaacaatctaccccttcacatctataccacttcttgcctactgtatccttccagccatctgtctccttactgacaaattcatcatgccaccgataagcacatttgctagtctcttcttcatctcactgtttatgtcgatcattgtaacgggaatcttggaattgagatggagcggagttagcattgaggaatggtggagaaacgagcaattctgggtcattggaggaatctcagctcatctctttgcggttgtccaaggtctcctcaaaatcttagcaggcattgacacaaacttcaccgtcacatcaaaggcaacagatgatgatgactttggagaactttacgcattcaaatggacaacactgctgatccctccaacaactgtcttaatcataaacattgttggcgttgttgcaggcatctcagatgccattaacaatggatatcagtcttggggacctctatttggtaaactcttcttctccttttgggtcattgttcatctctacccattcctcaaaggtctgatgggtagacagaacagaacaccaaccattgtggtgatttggtcagtgttattggcatctatcttctctttgctttgggtaagaattgatccttttgtgctcaagaccaaaggacctgacacttccaagtgtggcatcaactgctga

SEQ ID NO: 50 - protein, 1026 aa, synthetic construct
MEASAGLVAGSHNRNELVVIHNHEEPKPLKNLDGQFCEICGDQIGLTVEGDLFVACNECGFPACRPCYEYERREGTQNCPQCKTRYKRLRGSPRVEGDEDEEDIDDIEYEFNIEHEQDKHKHSAEAMLYGKMSYGRGPEDDENGRFPPVIAGGHSGEFPVGGGYGNGEHGLHKRVHPYPSSEAGSEGGWRERMDDWKLQHGNLGPEPDDDPEMGLIDEARQPLSRKVPIASSKINPYRMVIVVRLVILAVFLRYRLLNPVHDALGLWLTSVICEIWFAVSWILDQFPKWFPIERETYLDRLSLRYEREGEPNMLAPVDVFVSTVDPLKEPPLVTSNTVLSILAMDYPVEKISCYVSDDGASMLTFESLSETAEFARKWVPFCKKFSIEPRAPEMYFTLKVDYLQDKVHPTFVKERRAMKREYEEFKVRINAQVAKASKVPLEGWIMQDGTPWPGNNTKDHPGMIQVFLGHSGGFDVEGHELPRLVYVSREKRPGFQHHKKAGAMNALVRVAGVLTNAPFMLNLDCDHYVNNSKAVREAMCFLMDPQIGKKVCYVQFPQRFDGIDTNDRYANRNTVFFDINMKGLDGIQGPVYVGTGCVFKRQALYGYEPPKGPKRPKMISCGCCPCFGRRRKNKKFSKNDMNGDVAALGGAEGDKEHLMSEMNFEKTFGQSSIFVTSTLMEEGGVPPSSSPAVLLKEAIHVISCGYEDKTEWGTELGWIYGSITEDILTGFKMHCRGWRSIYCMPKRPAFKGSAPINLSDRLNQVLRWALGSVEIFFSRHSPLWYGYKGGKLKWLERFAYANTTIYPFTSIPLLAYCILPAICLLTDKFIMPPISTFASLFFISLFMSIIVTGILELRWSGVSIEEWWRNEQFWVIGGISAHLFAVVQGLLKILAGIDTNFTVTSKATDDDDFGELYAFKWTTLLIPPTTVLIINIVGVVAGISDAINNGYQSWGPLFGKLFFSFWVIVHLYPFLKGLMGRQNRTPTIVVIWSVLLASIFSLLWVRIDPFVLKTKGPDTSKCGINC

SEQ ID NO: 51 - DNA, 3081 bp, synthetic construct
  source 1..3081: mol_type = other DNA
atggaagctagcgccggtcttgtcgccggttctcataaccgtaatgaactagtcgtcattcacaaccatgaagagccaaagcctctgaagaatctagatggacaattctgtgagatatgtggagatcagattggtttaacagtagaaggagacctcttcgtagcttgcaatgagtgtggttttccggcgtgtagaccttgctatgagtacgagagaagagaaggaacacaaaactgtcctcagtgtaagactcgttacaagcgtctcagaggaagcccaagagtggagggagatgaaaacgaagaagatattgatgatattgagtatgaatttaatatcgaacatgaacaagataagcataagcattctgctgaggctatgctttatgggaaaatgagctatggaagaggtcctgaggatgatgagaatgggagattcccacctgttatagctggtggtcatagtggagaatttccagttggaggaggttatggtaatggagaacatgggcttcataagcgtgtgcacccatatccatcatctgaagctgggagtgagggaggatggcgggaaagaatggatgactggaagctccagcatggaaatcttgggccagaaccagatgatgatcctgagatgggactgatcgacgaggcacggcagccactctcgcggaaagttcccattgcctcaagcaagatcaatccatatcggatggtcatcgtcgctaggcttgtgattctagcagtttttctgcggtataggctcttgaatccagtgcatgatgctctgggattatggctgacctctgtgatctgtgaaatctggttcgctgtctcttggattcttgatcagttccccaagtggttccctattgaacgtgagacctatctagatcggctttccctcaggtacgagagagaaggtgaaccaaatatgcttgcccctgtagatgtctttgtcagtacggtggacccattgaaggagcctcccctcgtcacatccaacactgtgctgtcaatcttggccatggactacccagttgagaaaatctcctgctatgtctctgacgacggtgcttcaatgcttacattcgaatctctctcggaaactgctgagtttgcaagaaaatgggttcccttctgtaagaaattctccatagagccacgggcaccggagatgtacttcacgttgaaagttgattatctccaggacaaagtccacccaacatttgttaaggaacgtcgagccatgaagagagaatatgaggagttcaaggtcaggatcaacgctcaagtggcgaaggcctcaaaggttcctctagaaggttggatcatgcaagatggaacaccgtggccagggaacaacaccaaggaccaccccggtatgatccaagtcttcctcggccacagcggaggatttgatgtcgaagggcatgagcttcctcggcttgtgtacgtgtcccgtgagaagcgtcctggttttcaacaccacaagaaagctggcgccatgaatgccctggttcgagtggcaggcgtactcacaaatgctcctttcatgctgaacttggactgtgatcactatgtaaacaacagcaaggccgtgagggaagcaatgtgttttttgatggatcctcagattggaaagaaggtctgctatgttcagttccctcaaaggtttgatggcattgacacaaacgatcgttacgccaacagaaacacagtcttctttgatatcaatatgaaaggtctagatggaatccaaggtccagtttacgttggtactggttgtgttttcaaacgacaagctctgtatggttatgaaccaccaaagggtcctaaacgtccaaagatgataagctgtggttgttgtccttgctttgggcgccggagaaagaataagaaattttccaagaatgacatgaatggtgacgtagcagcccttggaggagcagaaggtgataaggagcatttgatgtctgaaatgaactttgagaaaacatttgggcaatcatccatctttgtaacctcaactttgatggaagaaggtggtgttcctccgtcatcaagtcctgcagtgctccttaaagaggcaatccatgtcataagctgcggttatgaagacaagactgagtggggaactgagctgggttggatctatggctctatcacagaggatattttgacgggattcaagatgcattgccgtggatggaggtctatttactgcatgcctaagaggcctgcattcaaaggttcagctcctattaatctatcagacaggttaaaccaggttttgcgttgggcacttggatcggttgagatatttttcagccggcacagtcctctctggtatggctacaaaggaggcaaactcaagtggcttgagcgttttgcttatgccaacacaacaatctaccccttcacatctataccacttcttgcctactgtatccttccagccatctgtctccttactgacaaattcatcatgccaccgataagcacatttgctagtctcttcttcatctcactgtttatgtcgatcattgtaacgggaatcttggaattgagatggagcggagttagcattgaggaatggtggagaaacgagcaattctgggtcattggaggaatctcagctcatctctttgcggttgtccaaggtctcctcaaaatcttagcaggcattgacacaaacttcaccgtcacatcaaaggcaacagatgatgatgactttggagaactttacgcattcaaatggacaacactgctgatccctccaacaactgtcttaatcataaacattgttggcgttgttgcaggcatctcagatgccattaacaatggatatcagtcttggggacctctatttggtaaactcttcttctccttttgggtcattgttcatctctacccattcctcaaaggtctgatgggtagacagaacagaacaccaaccattgtggtgatttggtcagtgttattggcatctatcttctctttgctttgggtaagaattgatccttttgtgctcaagaccaaaggacctgacacttccaagtgtggcatcaactgctga

SEQ ID NO: 52 - protein, 1026 aa, synthetic construct
MEASAGLVAGSHNRNELVVIHNHEEPKPLKNLDGQFCEICGDQIGLTVEGDLFVACNECGFPACRPCYEYERREGTQNCPQCKTRYKRLRGSPRVEGDENEEDIDDIEYEFNIEHEQDKHKHSAEAMLYGKMSYGRGPEDDENGRFPPVIAGGHSGEFPVGGGYGNGEHGLHKRVHPYPSSEAGSEGGWRERMDDWKLQHGNLGPEPDDDPEMGLIDEARQPLSRKVPIASSKINPYRMVIVARLVILAVFLRYRLLNPVHDALGLWLTSVICEIWFAVSWILDQFPKWFPIERETYLDRLSLRYEREGEPNMLAPVDVFVSTVDPLKEPPLVTSNTVLSILAMDYPVEKISCYVSDDGASMLTFESLSETAEFARKWVPFCKKFSIEPRAPEMYFTLKVDYLQDKVHPTFVKERRAMKREYEEFKVRINAQVAKASKVPLEGWIMQDGTPWPGNNTKDHPGMIQVFLGHSGGFDVEGHELPRLVYVSREKRPGFQHHKKAGAMNALVRVAGVLTNAPFMLNLDCDHYVNNSKAVREAMCFLMDPQIGKKVCYVQFPQRFDGIDTNDRYANRNTVFFDINMKGLDGIQGPVYVGTGCVFKRQALYGYEPPKGPKRPKMISCGCCPCFGRRRKNKKFSKNDMNGDVAALGGAEGDKEHLMSEMNFEKTFGQSSIFVTSTLMEEGGVPPSSSPAVLLKEAIHVISCGYEDKTEWGTELGWIYGSITEDILTGFKMHCRGWRSIYCMPKRPAFKGSAPINLSDRLNQVLRWALGSVEIFFSRHSPLWYGYKGGKLKWLERFAYANTTIYPFTSIPLLAYCILPAICLLTDKFIMPPISTFASLFFISLFMSIIVTGILELRWSGVSIEEWWRNEQFWVIGGISAHLFAVVQGLLKILAGIDTNFTVTSKATDDDDFGELYAFKWTTLLIPPTTVLIINIVGVVAGISDAINNGYQSWGPLFGKLFFSFWVIVHLYPFLKGLMGRQNRTPTIVVIWSVLLASIFSLLWVRIDPFVLKTKGPDTSKCGINC

SEQ ID NO: 53 - DNA, 2958 bp, Arabidopsis thaliana
  source 1..2958: mol_type = other DNA
atgatggagtctaggtctcccatctgcaacacttgtggtgaagagattggtgtaaaatcaaacggagagttctttgtggcttgtcatgagtgtagtttcccgatctgcaaagcttgtcttgagtatgaattcaaagaaggtcgaagaatttgcttgcgttgcggcaatccttacgatgagaatgtgtttgatgatgttgagacaaagacatctaaaactcaatccattgttccaacacagaccaataacacttctcaggattcagggattcatgctagacatataagtacagtctcaacaatagacagtgaactgaatgatgaatatggcaatccaatttggaagaacagagtggagagctggaaagacaagaaagacaagaagagcaagaagaagaagaaagatccaaaagcaacaaaagctgaacaacatgaggctcagattcctacccaacagcacatggaagatacgccaccgaacacagaatctggtgctacagatgtgctttcggttgtgattcctatcccaaggacaaaaatcacttcatataggattgtcatcatcatgcggttgatcatcttggctctgttttttaactaccgtatcacgcatcctgtcgatagcgcttacggtttatggctaacatctgtgatatgtgagatttggtttgctgtttcttgggtgttggatcagttccctaaatggtctcctattaaccgagaaacttacatcgaccggttatccgcaagattcgaaagagaaggcgagcaatcacagcttgcagctgtagatttctttgttagtacggtagatccattaaaggagccacctttgataactgcaaacacggttctttcgatcctcgcgcttgattatccggtggataaagtctcttgctatgtatctgatgatggtgctgcaatgctttcgtttgagtctttggttgagacagcagattttgctaggaaatgggtacctttctgcaaaaagtactccatcgagccacgagctcccgagttttacttctcgcttaaaatcgattacttgagggataaagttcaaccttcttttgtgaaagaacgtagagccatgaaaagagattatgaagagtttaaaataagaatgaatgctttagtcgccaaggctcaaaagacaccagaagaaggatggacaatgcaagatggaacatcttggcccgggaacaacactcgtgaccatcccgggatgattcaggtttttcttggatatagcggtgctcgcgacattgaaggaaatgaacttccaagattagtttacgtctctagagagaagagacctggttatcagcatcacaaaaaggccggggcagagaacgcattggtgagggtgtctgcggttttaacgaatgctccattcattcttaaccttgattgtgatcactacgtcaacaatagcaaagccgtgcgtgaagcaatgtgctttttaatggatcctgttgttggtcaagacgtttgctttgttcagttcccacagagatttgatggaatcgacaagagtgatcgatatgctaaccgcaacattgttttcttcgatgttaatatgagagggcttgatgggattcaaggtccagtttatgttggtacaggtactgtctttagaagacaagcactttacggatacagtccaccttcaaaaccgaggattttaccgcaatcttcatcatcgtcgtgttgctgtctaaccaagaagaaacaacctcaagatccttccgagatttacaaagatgcaaagcgagaagaacttgatgctgcaatctttaatcttggggacctagacaactacgatgagtacgacagatcgatgctgatttcacaaacaagctttgagaaaacgtttggtctctctacggttttcatcgagtctactcttatggagaatggcggtgttcccgactctgtaaacccgtcaacgctcatcaaagaagctattcatgtcattagctgtggatacgaagagaaaactgaatggggaaaagaaataggatggatttacgggtcgatcaccgaagacattttgacgggtttcaagatgcattgtcgtggatggaggtcgatttactgtatgccattaagaccagcatttaaaggatctgctccaatcaatctatcagataggcttcaccaggttctacgttgggctcttggctcggttgagatcttccttagccgacattgtcctttgtggtacggttgcagcggaggccgcctcaagttgctccagagattagcttatataaacactattgtctacccattcacttctttgcctcttgttgcttactgtactcttccagctatttgccttcttaccggcaaatttatcatcccaacgctatcaaacctagcaagcatgctgtttctaggtctctttatatcaatcatcttaacgagtgtcctcgagcttcgatggagcggagtcagtatcgaagacttatggagaaacgaacagttttgggttattggaggtgtctcagctcatctctttgccgttttccaaggattcctcaaaatgctcgctggtctcgacacaaatttcacagtcacatcgaaaaccgcagatgatttagaattcggtgagctttacattgtcaaatggacaactctcttgatccctccgacgtcacttcttataatcaacttggtcggagttgttgctggattctctgacgctcttaacaaaggttatgaagcttggggacctttgtttgggaaggtatttttcgccttttgggtgattcttcatctttatccattcctcaaaggtcttatgggaagacaaaacagaacacctactattgttattctctggtctatcttgcttgcttctgtgttttcacttgtttgggttcgtatcaatcctttcgtctccaaaaccgatacgacttccctttctctgaactgtcttttgatcgattgctaa

SEQ ID NO: 54 - protein, 985 aa, Arabidopsis thaliana
MMESRSPICNTCGEEIGVKSNGEFFVACHECSFPICKACLEYEFKEGRRICLRCGNPYDENVFDDVETKTSKTQSIVPTQTNNTSQDSGIHARHISTVSTIDSELNDEYGNPIWKNRVESWKDKKDKKSKKKKKDPKATKAEQHEAQIPTQQHMEDTPPNTESGATDVLSVVIPIPRTKITSYRIVIIMRLIILALFFNYRITHPVDSAYGLWLTSVICEIWFAVSWVLDQFPKWSPINRETYIDRLSARFEREGEQSQLAAVDFFVSTVDPLKEPPLITANTVLSILALDYPVDKVSCYVSDDGAAMLSFESLVETADFARKWVPFCKKYSIEPRAPEFYFSLKIDYLRDKVQPSFVKERRAMKRDYEEFKIRMNALVAKAQKTPEEGWTMQDGTSWPGNNTRDHPGMIQVFLGYSGARDIEGNELPRLVYVSREKRPGYQHHKKAGAENALVRVSAVLTNAPFILNLDCDHYVNNSKAVREAMCFLMDPVVGQDVCFVQFPQRFDGIDKSDRYANRNIVFFDVNMRGLDGIQGPVYVGTGTVFRRQALYGYSPPSKPRILPQSSSSSCCCLTKKKQPQDPSEIYKDAKREELDAAIFNLGDLDNYDEYDRSMLISQTSFEKTFGLSTVFIESTLMENGGVPDSVNPSTLIKEAIHVISCGYEEKTEWGKEIGWIYGSITEDILTGFKMHCRGWRSIYCMPLRPAFKGSAPINLSDRLHQVLRWALGSVEIFLSRHCPLWYGCSGGRLKLLQRLAYINTIVYPFTSLPLVAYCTLPAICLLTGKFIIPTLSNLASMLFLGLFISIILTSVLELRWSGVSIEDLWRNEQFWVIGGVSAHLFAVFQGFLKMLAGLDTNFTVTSKTADDLEFGELYIVKWTTLLIPPTSLLIINLVGVVAGFSDALNKGYEAWGPLFGKVFFAFWVILHLYPFLKGLMGRQNRTPTIVILWSILLASVFSLVWVRINPFVSKTDTTSLSLNCLLIDC

SEQ ID NO: 55 - DNA, 2958 bp, synthetic construct
  source 1..2958: mol_type = other DNA
atgatggagtctaggtctcccatctgcaacacttgtggtgaagagattggtgtaaaatcaaacggagagttctttgtggcttgtcatgagtgtagtttcccgatctgcaaagcttgtcttgagtatgaattcaaagaaggtcgaagaatttgcttgcgttgcggcaatccttacgatgagaatgtgtttgatgatgttgagacaaagacatctaaaactcaatccattgttccaacacagaccaataacacttctcaggattcagggattcatgctagacatataagtacagtctcaacaatagacagtgaactgaatgatgaatatggcaatccaatttggaagaacagagtggagagctggaaagacaagaaagacaagaagagcaagaagaagaagaaagatccaaaagcaacaaaagctgaacaacatgaggctcagattcctacccaacagcacatggaagatacgccaccgaacacagaatctggtgctacagatgtgctttcggttgtgatttctatcccaaggacaaaaatcacttcatataggattgtcatcatcatgcggttgatcatcttggctctgttttttaactaccgtatcacgcatcctgtcgatagcgcttacggtttatggctaacatctgtgatatgtgagatttggtttgctgtttcttgggtgttggatcagttccctaaatggtctcctattaaccgagaaacttacatcgaccggttatccgcaagattcgaaagagaaggcgagcaatcacagcttgcagctgtagatttctttgttagtacggtagatccattaaaggagccacctttgataactgcaaacacggttctttcgatcctcgcgcttgattatccggtggataaagtctcttgctatgtatctgatgatggtgctgcaatgctttcgtttgagtctttggttgagacagcagattttgctaggaaatgggtacctttctgcaaaaagtactccatcgagccacgagctcccgagttttacttctcgcttaaaatcgattacttgagggataaagttcaaccttcttttgtgaaagaacgtagagccatgaaaagagattatgaagagtttaaaataagaatgaatgctttagtcgccaaggctcaaaagacaccagaagaaggatggacaatgcaagatggaacatcttggcccgggaacaacactcgtgaccatcccgggatgattcaggtttttcttggatatagcggtgctcgcgacattgaaggaaatgaacttccaagattagtttacgtctctagagagaagagacctggttatcagcatcacaaaaaggccggggcagagaacgcattggtgagggtgtctgcggttttaacgaatgctccattcattcttaaccttgattgtgatcactacgtcaacaatagcaaagccgtgcgtgaagcaatgtgctttttaatggatcctgttgttggtcaagacgtttgctttgttcagttcccacagagatttgatggaatcgacaagagtgatcgatatgctaaccgcaacattgttttcttcgatgttaatatgagagggcttgatgggattcaaggtccagtttatgttggtacaggtactgtctttagaagacaagcactttacggatacagtccaccttcaaaaccgaggattttaccgcaatcttcatcatcgtcgtgttgctgtctaaccaagaagaaacaacctcaagatccttccgagatttacaaagatgcaaagcgagaagaacttgatgctgcaatctttaatcttggggacctagacaactacgatgagtacgacagatcgatgctgatttcacaaacaagctttgagaaaacgtttggtctctctacggttttcatcgagtctactcttatggagaatggcggtgttcccgactctgtaaacccgtcaacgctcatcaaagaagctattcatgtcattagctgtggatacgaagagaaaactgaatggggaaaagaaataggatggatttacgggtcgatcaccgaagacattttgacgggtttcaagatgcattgtcgtggatggaggtcgatttactgtatgccattaagaccagcatttaaaggatctgctccaatcaatctatcagataggcttcaccaggttctacgttgggctcttggctcggttgagatcttccttagccgacattgtcctttgtggtacggttgcagcggaggccgcctcaagttgctccagagattagcttatataaacactattgtctacccattcacttctttgcctcttgttgcttactgtactcttccagctatttgccttcttaccggcaaatttatcatcccaacgctatcaaacctagcaagcatgctgtttctaggtctctttatatcaatcatcttaacgagtgtcctcgagcttcgatggagcggagtcagtatcgaagacttatggagaaacgaacagttttgggttattggaggtgtctcagctcatctctttgccgttttccaaggattcctcaaaatgctcgctggtctcgacacaaatttcacagtcacatcgaaaaccgcagatgatttagaattcggtgagctttacattgtcaaatggacaactctcttgatccctccgacgtcacttcttataatcaacttggtcggagttgttgctggattctctgacgctcttaacaaaggttatgaagcttggggacctttgtttgggaaggtatttttcgccttttgggtgattcttcatctttatccattcctcaaaggtcttatgggaagacaaaacagaacacctactattgttattctctggtctatcttgcttgcttctgtgttttcacttgtttgggttcgtatcaatcctttcgtctccaaaaccgatacgacttccctttctctgaactgtcttttgatcgattgctaa

SEQ ID NO: 56 - protein, 985 aa, synthetic construct
MMESRSPICNTCGEEIGVKSNGEFFVACHECSFPICKACLEYEFKEGRRICLRCGNPYDENVFDDVETKTSKTQSIVPTQTNNTSQDSGIHARHISTVSTIDSELNDEYGNPIWKNRVESWKDKKDKKSKKKKKDPKATKAEQHEAQIPTQQHMEDTPPNTESGATDVLSVVISIPRTKITSYRIVIIMRLIILALFFNYRITHPVDSAYGLWLTSVICEIWFAVSWVLDQFPKWSPINRETYIDRLSARFEREGEQSQLAAVDFFVSTVDPLKEPPLITANTVLSILALDYPVDKVSCYVSDDGAAMLSFESLVETADFARKWVPFCKKYSIEPRAPEFYFSLKIDYLRDKVQPSFVKERRAMKRDYEEFKIRMNALVAKAQKTPEEGWTMQDGTSWPGNNTRDHPGMIQVFLGYSGARDIEGNELPRLVYVSREKRPGYQHHKKAGAENALVRVSAVLTNAPFILNLDCDHYVNNSKAVREAMCFLMDPVVGQDVCFVQFPQRFDGIDKSDRYANRNIVFFDVNMRGLDGIQGPVYVGTGTVFRRQALYGYSPPSKPRILPQSSSSSCCCLTKKKQPQDPSEIYKDAKREELDAAIFNLGDLDNYDEYDRSMLISQTSFEKTFGLSTVFIESTLMENGGVPDSVNPSTLIKEAIHVISCGYEEKTEWGKEIGWIYGSITEDILTGFKMHCRGWRSIYCMPLRPAFKGSAPINLSDRLHQVLRWALGSVEIFLSRHCPLWYGCSGGRLKLLQRLAYINTIVYPFTSLPLVAYCTLPAICLLTGKFIIPTLSNLASMLFLGLFISIILTSVLELRWSGVSIEDLWRNEQFWVIGGVSAHLFAVFQGFLKMLAGLDTNFTVTSKTADDLEFGELYIVKWTTLLIPPTSLLIINLVGVVAGFSDALNKGYEAWGPLFGKVFFAFWVILHLYPFLKGLMGRQNRTPTIVILWSILLASVFSLVWVRINPFVSKTDTTSLSLNCLLIDC

(19) United States
(12) Patent Application Publication
(10) Pub. No.: US 2025/0263368 A1
(43) Pub. Date: Aug. 21, 2025

(54) CELLULOSE SYNTHASE INHIBITORS AS A NEW CLASS OF HERBICIDE AND NON-GMO CROPS THAT ARE RESISTANT TO THE HERBICIDE

(71) Applicant: Purdue Research Foundation, West Lafayette, IN (US)

(72) Inventors: Chunhua Zhang, West Lafayette, IN (US); Lei Huang, West Lafayette, IN (US); Xiaohui Li, West Lafayette, IN (US)

(21) Appl. No.: 19/204,442

(22) Filed: May 9, 2025

Related U.S. Application Data
(63) Continuation of application No. 16/765,788, filed on May 20, 2020.
(60) Provisional application No. 62/588,677, filed on Nov. 20, 2017.

(51) Int. Cl. C07C 335/40; A01N 47/34; C12N 9/10

(52) U.S. Cl. CPC C07C 335/40; A01N 47/34; C12N 9/1059

(57) ABSTRACT

Using chemical genetic screening, we discovered a small molecule Cellulosin (aka endosidin20 or ES20) that causes cell swollen and inhibits plant growth, but does not disrupt global vesicle trafficking. By doing mutant screening, we obtained multiple alleles of Arabidopsis thaliana that are resistant to Cellulosin inhibition in growth. Those mutated amino acid residues are conserved across plant species. Cellulosin targets a group of cellulose synthases (CesAs) of Arabidopsis thaliana by binding to a conserved domain essential for the catalytic activity of CesA. Cellulosin may target and inhibit all subtypes of CesAs in plants. The present invention relates to Cellulosin, a cellulose synthase inhibitor, its analogs or derivatives as a broad-spectrum herbicide. The mutated genes, their protein products and a cell or a plant having those mutated genes or expressing those protein products are within the scope of this disclosure.

### CROSS-REFERENCE TO RELATED APPLICATIONS

The present U.S. patent application relates to and claims the priority benefit of U.S. Provisional Patent Application Ser. No. 62/588,677, filed Nov. 20, 2017, the content of which is hereby incorporated by reference in its entirety.

### STATEMENT OF SEQUENCE LISTING

A computer-readable form (CRF) of the Sequence Listing is submitted concurrently with this application. The file, generated on Nov. 19, 2018, is entitled Sequence_Listing_68083-02_ST25_txt. Applicant states that the content of the computer-readable form is the same and the information recorded in computer readable form is identical to the written sequence listing.

### TECHNICAL FIELD

The present application relates to composition matters of cellulose synthase inhibitors as a herbicide and mutant genes that convey resistance to those herbicides to a plant that expresses those mutant genes.

### BACKGROUNDS AND SUMMARY OF THE INVENTION

This section introduces aspects that may help facilitate a better understanding of the disclosure. Accordingly, these statements are to be read in this light and are not to be understood as admissions about what is or is not prior art.

Plant cell wall determines plant cell morphology and is the major composition of plant biomass. Cellulose is the polymer of β(1,4) D-glucose and is an essential cell wall component that controls anisotropic plant cell growth. Cellulose is the most abundant biopolymers on earth and provides clothes, food, shelter and energy for human. Although the composition of cellulose is only the linear chain of β(1,4) D-glucose units, its mechanisms of biosynthesis in plants is extremely complicated but poorly understood. Cellulose microfibrils are synthesized by cellulose synthase complexes (CSCs) that move along the plasma membrane through the direction of underlying cortical microtubules (Bashline, L. et al., Plant Physiol. 2013, 163, 150-160; Bashline, L., et al., Proc. Natl Acad Sci. USA 2015, 112, 12870-12875; Parredez, A. R. et al., Science 2006, 312, 1491-1495). CSCs are assembled in the Golgi and delivered to the plasma membrane via small secretory vesicles (Crowell, E. F. et al., Plant Cell 2009, 21, 1141-1154; Gutierrez, R., et al., Nat. Cell Biol. 2009, 11, 797-806). Although the rosette structured CSCs were first observed from freeze fractured maize cells almost forty years ago and plant CesA genes were first cloned from cotton fiber twenty years ago, there are still many knowledge gaps regarding the mechanisms of plant cellulose biosynthesis.

For example, the mechanisms of spatiotemporal control of CSC trafficking to the plasma membrane, the mechanisms of CSC catalyzing β(1,4) D-glucose polymerization, the interaction between CSC and other proteins and lipids on the plasma membrane and the mechanisms of integration between cellulose polymerization and anisotropic cell growth. CSC is composed of cellulose synthase (CesA) proteins (the number of cellulose synthase protein per complex is still under debate in the cell wall field) that have multiple transmembrane domains and are extremely challenging to manipulate in vitro. A good amount of knowledge about plant cellulose synthesis came from phenotypic analysis of different cesa mutants that carry missense mutations (Slabaugh, E. et al., Trends Plant Sci. 2014, 19, 99-106; Kumar, M. et al., Phytochemistry, 2015, 112, 91-99). Small molecules have also been helpful in understanding cellulose synthesis because some of the missense mutants were identified through chemical genetic screens, although the mechanisms of action of most of these small molecules are not known (Tateno, M. et al., J Exp Bot 2016, 67, 533-542). A collection of inhibitors that could target different domains of CesAs will greatly facilitate in understanding the function of these domains.

Using chemical genetic screen in Arabidopsis thaliana, we identified a small molecule Endosidin20 (ES20) that reduces plant growth and inhibits anisotropic cell expansion. Our preliminary data show that ES20 affects CSC trafficking at the plasma membrane and physically interacts with the CesA6 subunit of the CSC. Structure modeling and molecular docking experiments indicate that ES20 could bind to a conserved pocket in the catalytic domain of CesAs. In a ES20-suppression screen, we found seven mutant alleles of Arabidopsis CesA6 and one mutant allele of CesA7 that are less sensitive to ES20, but did not find mutants in other CesA genes expressed during primary wall formation. The seven mutant alleles of cesa6 contain mis-sense mutations in amino acids located in the first central cytoplasmic catalytic domain and a poorly characterized smaller second cytoplasmic domain. The one cesa7 allele is located at the N-terminal cytoplasmic region. Our small molecule, live cell imaging tools, protein structure and molecular docking approaches and availability of a group of newly identified different mutant alleles place us in a unique position in understanding cellulose synthase trafficking, cellulose biosynthesis and their relationship to cell growth. In this proposal, we aim to understand the integration of cellulose synthesis and cell growth control by studying the function of seven amino acids that are essential for plants' sensitivity to ES20. We hypothesize that ES20 targets Arabidopsis thaliana CesAs, especially CesA6 and CesA7, at the core catalytic domain to affect cellulose synthesis. Amino acids at the core catalytic domain contribute different roles in cellulose biosynthesis and cell shape control. We test our hypothesis using the combination of chemical genetics, live cell imaging, cell wall analysis, biochemical analysis and plant phenotypic characterization.

Using chemical genetic screening, we discovered a small molecule Cellulosin (aka endosidin20 or ES20) that causes cell swollen and inhibits plant growth, but does not disrupt global vesicle trafficking. By doing mutant screening, we obtained multiple alleles of Arabidopsis thaliana that are resistant to Cellulosin inhibition in growth. Those mutated amino acid residues are conserved across plant species. Cellulosin targets a group of cellulose synthases (CesAs) of Arabidopsis thaliana by binding to a conserved domain essential for the catalytic activity of CesA. Cellulosin may target and inhibit all subtypes of CesAs in plants. The present invention relates to Cellulosin, a cellulose synthase inhibitor, its analogs or derivatives as a broad-spectrum herbicide. The mutated genes, their protein products and a cell or a plant having those mutated genes or expressing those protein products are within the scope of this disclosure.

### BRIEF DESCRIPTION OF THE DRAWINGS

A better understanding of the present invention will be obtained upon reference to the following description in conjunction with the accompanying drawings.

FIGS. 1A-1J depict ES20 inhibits anisotropic plant cell growth and reduces crystallize cellulose content in dark-grown seedlings. FIG. 1A shows the molecular structure of ES20. FIG. 1B shows ES20 inhibits Arabidopsis root growth and causes root swollen in a dosage-dependent manner. Arabidopsis wildtype seedlings grown on different concentrations of ES20 are shown. FIG. 1C. Growth curve of Arabidopsis wildtype plants grown on different concentrations of ES20. FIG. 1D. The tip region of 5 days old Arabidopsis roots treated with DMSO or 3 μM ES20 for 12 hours. Seedlings treated with ES20 have swollen roots in compare with DMSO control. FIG. 1E. The epidermal cells of 5 days old Arabidopsis roots treated with 3 μM ES20 for 12 hours are swollen in compare with DMSO control. FIG. 1F. ES20 inhibits Arabidopsis hypocotyl growth and cause hypocotyl swollen in a dosage dependent manner. Arabidopsis wildtype seedlings grown on different concentrations of ES20 under dark condition are shown. FIG. 1G. Quantification on the length of 5 days old dark grown hypocotyls in the presence of different concentrations of ES20. FIG. 1H. Hypocotyls from seedlings grown on ES20 media under dark condition are wider in compare with seedlings grown on DMSO control. FIG. 1I. The hypocotyl epidermal cells of plants grown in the presence of ES20 under dark condition are swollen in compare with DMSO control. J. ES20 reduces the content of crystalline cellulose in dark-grown Arabidopsis seedlings in a dosage dependent manner. If there is no common letter between two groups, it indicates they are significant different in crystalline cellulose content by paired t-test, p<0.05. Scale bars in FIGS. 1B and 1F are 1.0 cm; Bars in FIGS. 1D and 1H are 100 μm. Scale bars in FIGS. 1E and 1I are 20 μm.

FIGS. 2A-2F depict a group of cesa6 that have reduced sensitivity to ES20. FIG. 2A. 6days old seedlings of wildtype (WT) and cesa6 mutants (es20r1 to es20r10) that were grown on growth media supplemented with DMSO solvent control. FIG. 2B. 6 days old seedlings of wildtype and cesa6 alleles grown on growth media supplemented with 1 μM ES20. FIG. 2C. Quantification on the root length of 6 days old wildtype and mutant seedlings grown on growth media supplemented with DMSO solvent control. FIG. 2D. Quantification on the root length of wildtype and mutant seedlings grown on growth media supplemented with 1 μM ES20. FIG. 2E. The ratio of root length from plants grown on 1 μM ES20 and those grown on DMSO for each genotype. The root length of wildtype plants grown on 1 μM ES20 was reduced to about 25% of those grown on DMSO. However, in some mutants, the root growth was not reduced at all (the ratio was close to 1) by 1 μM ES20 or was slightly reduced but was not as significant as that of the wildtype plants. FIG. 2F. The diagram shows the topology of CesA6, the location, and the mutated amino acid in each mutant allele.

FIGS. 3A-3C demonstrate that ES20 directly interacts with CesA6 at the catalytic core. FIG. 3A. The thermophoresis binding curve for the interaction of CesA6 central cytoplasmic domain with different concentrations of ES20. FIG. 3B. The thermophoresis binding curve for the interaction of CesA6 central cytoplasmic domain with different concentrations of UDP-glucose. FIG. 3C. Hypothesized binding site for ES20 (cyan) and UDP-glucose (magenta) on modeled structure of CesA6 central cytoplasmic domain. The amino acids highlighted by green indicate the ones found to have been mutated in our reduced sensitivity mutants. The amino acids highlighted by yellow indicate the ones that we mutated based on hypothesized binding site and have caused reduced sensitivity to ES20 in plants.

FIG. 4. Mutations potentially reduce sensitivity of a plant to ES20 and transgenic plants that express mutated CesA6 in prc1-1 genetic background have reduced sensitivity to ES20.

FIGS. 5A-5E show that ES20 treatment inhibits exocytic trafficking of CesA6. FIGS. 5A and 5B. The density of plasma membrane localized CSCs labeled by YFP-CesA6 was significantly reduced by ES20 treatment. FIGS. 5C and 5D. The numbers of SmaCCs were significantly increased by ES20 treatment. FIGS. 5E and 5F. The exocytic delivery of CSC to the plasma membrane was reduced after ES20 treatment. *** indicates p<0.001 by paired t-test.

FIGS. 6A-6F demonstrate that ES20 targets multiple CesAs in plants. Plants that carry a P557T mutation in CesA7 (fra5) and plants carry a D604N mutation in CesA1 (any1) are less sensitive to ES20. FIG. 6A. Seedlings of Wildtype and fra5 grown on growth media supplemented with DMSO or ES20. FIG. 6B. Seedlings of Wildtype and any1 grown on growth media supplemented with DMSO or ES20. FIGS. 6C, 6D, 6E and 6F depict quantification on the growth of wildtype, fra1 and any1 in the presence or absence of ES20.

FIGS. 7A-7C show that ES20 analog 3 is more potent than ES20 in inhibiting plant growth. FIG. 7A. Molecular structures of ES20 and analogs. FIG. 7B. The effect of ES20 and its analogs on Arabidopsis root growth. 7-day-old Arabidopsis seedlings grown endoc Cellulosin and analogs are shown. Bars, 1 cm. FIG. 7C. Quantification on the effects of Cellulosin and analogs on Arabidopsis root growth.

FIGS. 8A-8B demonstrate that ES20 is active in inhibiting tomato and horseweed growth. FIG. 8A shows 6-day-old tomato and horseweed grown on the media in the absence and presence of Cellulosin. FIG. 8B depicts 16-day-old tomato and horseweed grown on the media in the absence and presence of Cellulosin. Bars, 1 cm.

FIGS. 9A and 9B demonstrate that ES20 does not disrupt general endomembrane system of plants. Transgenic plants expressing different fluorescence-tagged proteins were examined for their cellular localization in the DMSO control treatment and after 2 hours of 5 μM ES20 treatment.

FIGS. 10A, 10B and 10C depict expression of CesA6 with missense mutations in prc1-1 background is sufficient to create plants with reduced sensitivity to ES20.

FIG. 11A shows locations of point mutations in our cesa6 alleles. The sequence alignment was done by Slabaugh et al. (Slabaugh, D., et al., Trends Plant Sci 2014, 19, 99-106) and was adopted here to show the locations of mutations: Gray shade indicates predicted transmembrane helices, blue shade indicates BcsA interfacial helixes, orange circles denote the locations of known missense mutations in AtCESAs. Orange squares are the mutations we discovered.

FIG. 11B shows comparison of seedling growth between wildtype and different mutants on DMSO and Cellulosin. The mutation in each mutant is labeled. All mutants are alleles of cesa6 except D100N that is a mutant of cesa7.

FIG. 11C shows demonstrates expression of mutant CesA construct in wildtype plants leads to resistance to ES20. The root length of wildtype plants grown on 1 μM ES20 is about 20% of those that are grown on normal media.

FIG. 11D shows root growth of mutants CesA6 and the wildtype in the presence of 1 μM of ES20 or DMSO as a control. The root length of cesa6E929k mutant and transgenic plants that express CesA6F929K in wildtype is about 80% of those grown on normal media.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 20%, within 10%, within 5%, or within 1% of a stated value or of a stated limit of a range. In the present disclosure, the term “substantially” can allow for a degree of variability in a value or range, for example, within 80%, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

“Identity,” as is well understood in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. Methods to determine “identity” are designed to give the largest match between the sequences tested. Moreover, methods to determine identity are codified in publicly available programs. Computer programs can be used to determine “identity” between two sequences these programs include but are not limited to, GCG; suite of five BLAST programs, three designed for nucleotide sequences queries (BLASTN, BLASTX, and TBLASTX) and two designed for protein sequence queries (BLASTP and TBLASTN). The BLAST X program is publicly available from NCBI and other sources. The well-known Smith Waterman algorithm can also be used to determine identity.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as 13 CH═CH—, —CH═CHCH₂—, and the like. Examples of branched alkenyl groups include, but are not limited to, —CH═C(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl) alkyl groups such as 4-ethyl-indanyl. Aralkenyl groups are alkenyl groups as defined herein in which a hydrogen or carbon bond of an alkyl group is replaced with a bond to an aryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastercomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 g/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

It is understood that, the herbicides disclosed herein can be applied to a field of a plant for weed control at the same time as a pre-formulated mixture, or applied individually as a separately pre-formulated product, consequentially or concurrently.

It is understood that, multiple application of said composition of herbicides may be needed in some cases in order achieve effective and efficient weed control for a field of a plant. As disclosed herein said plant is resistant to the herbicides applied.

In preparing a product for an end user, adjuvants, surfactants, anti-drifting agents, colorings, anti-freezing or other stabilizing chemicals may be included. An adjuvant is an additive (usually in relatively low amounts compared to the carrier) that improves or enhances application, performance, safety, storage, or handling of an active ingredient. Adjuvants include materials such as: Surfactants (spreaders, stickers, emulsifiers, wetting agents), which increase surface contact, reduce runoff, and increase penetration through leaf cuticle.

Pure herbicide molecules have to be properly formulated before it is delivered to the end user for various purposes of weed control and rarely used as the pure chemical. In addition, a given chemical may be formulated in a variety of differing formulations and sold under different trade names. The primary reason for formulating a herbicide is to allow the user to dispense it in a convenient carrier, such as water. The primary purpose of the carrier is to enable the uniform distribution of a relatively small amount of herbicide over a comparatively large area. In addition to providing the consumer with a form of herbicide that is easy to handle, formulating a herbicide can enhance the phytotoxicity of the herbicide, improve the shelf-life (storage) of the herbicide, and protect the herbicide from adverse environmental conditions while in storage or transit. Formulations vary according to the solubility of the herbicide active ingredient in water, oil and organic solvents, and the manner the formulation is applied (i.e., dispersed in a carrier such as water or applied as a dry formulation itself) (P. Miller and P. Westra, Colorado State University, www.colostate.edu).

Solution (S)—Solution formulations are designed for those active ingredients that dissolve readily in water. The formulation is a liquid and consists of the active ingredient and additives. When herbicides formulated as solutions are mixed with water, the active ingredient will not settle out of solution or separate.

Soluble Powder (SP)—Soluble powder formulations are similar to Solutions(S) in that, when mixed with water, these dry formulations dissolve readily and form a true solution. The formulation is dry and consists of the active ingredient and additives. When thoroughly mixed, no further agitation is necessary to keep the active ingredient dissolved in solution. Few formulations of this type are available because few active ingredients are highly soluble in water.

Emulsifiable Concentrate (E or EC)—Formulations of this type are liquids that contain the active ingredient, one or more solvents, and an emulsifier that allows mixing with water. Formulations of this type are highly concentrated and relatively inexpensive per pound of active ingredient; easy to handle, transport, and store; require little agitation (will not settle out or separate); and are not abrasive to machinery or spraying equipment. Formulations of this type may, however, have potentially greater phytotoxicity than other formulations; exhibit a potential for over-or under-dosing through mixing or calibration errors; are more easily absorbed through skin of humans or animals; and contain solvents that may cause deterioration of rubber or plastic hoses and pump parts.

Wettable Powder (W or WP)—Wettable powders are dry, finely ground formulations in which the active ingredient is combined with a finely ground carrier (usually mineral clay) along with other ingredients, to enhance the ability of the active ingredient plus carrier to suspend in water. The powder is mixed with water for application. Wettable powders are one of the most widely used herbicide formulations and offer low cost and ease of storage, transport, and handling; lower phytotoxicity potential than ECs and other liquid formulations; and less skin and eye absorption hazard than ECs and other liquid formulations. Some disadvantages are that they require constant and thorough agitation in the spray tank, are abrasive to pumps and nozzles (causing premature wear), may produce visible residues on plant and soil surfaces, and can create an inhalation hazard to the applicator while handling (pouring and mixing) the concentrated powder.

Liquid Flowable (F or FL)—Liquid flowable formulations consist of finely ground active ingredient suspended in a liquid. Flowables are mixed with water for application, are easily handled and applied, and seldom clog nozzles. Some of their disadvantages are that they may leave a visible residue on plant and soil surfaces, and typically require constant and thorough agitation to remain in suspension.

Dry Flowables and Water-Dispersible Granules (DF, DG or WDG)—Dry flowable and water-dispersible granule formulations are much like wettable powders except that the active ingredient is formulated on a large particle (granule) instead of onto a ground powder. This type of formulation offers essentially the same advantages and disadvantages as wettable powder formulations. However, these formulations generally are more easily mixed and measured than wettable powders. Because they create less dust when handling, they cause less inhalation hazard to the applicator during pouring and mixing.

Granules and Pellets (G, P or PS)—Used exclusively for soil applied herbicides, the active ingredient is formulated onto large particles (granules or pellets). The primary advantages of this type of formulation are that the formulation is ready to use with simple application equipment (seeders or spreaders), and the drift potential is low because the particles are large and settle quickly. The disadvantages of these formulations are that they do not adhere to foliage (not intended for foliar applications), and may require mixing into the soil in order to achieve adequate herbicidal activity.

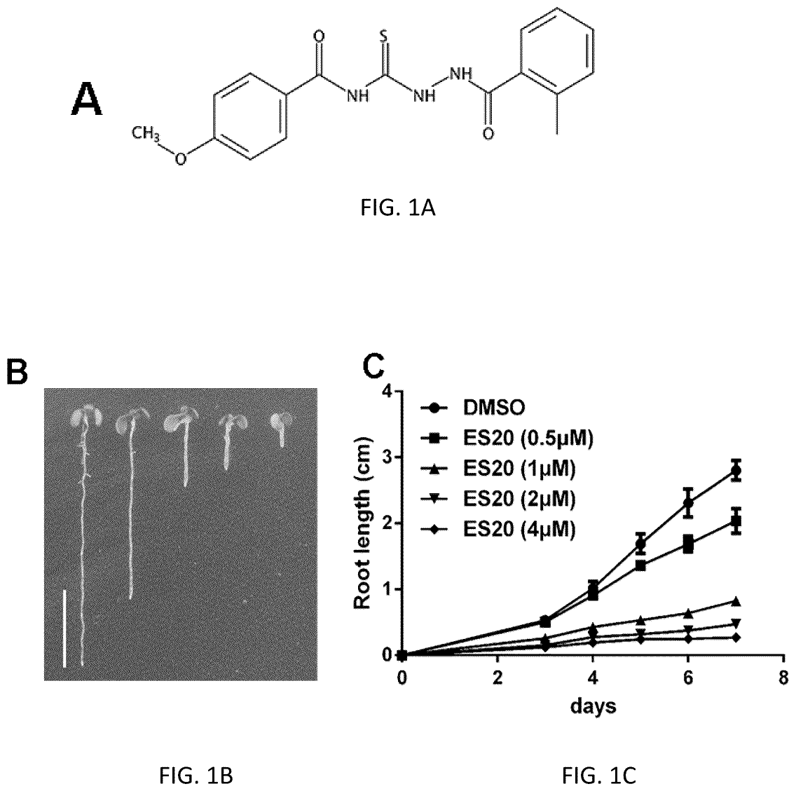

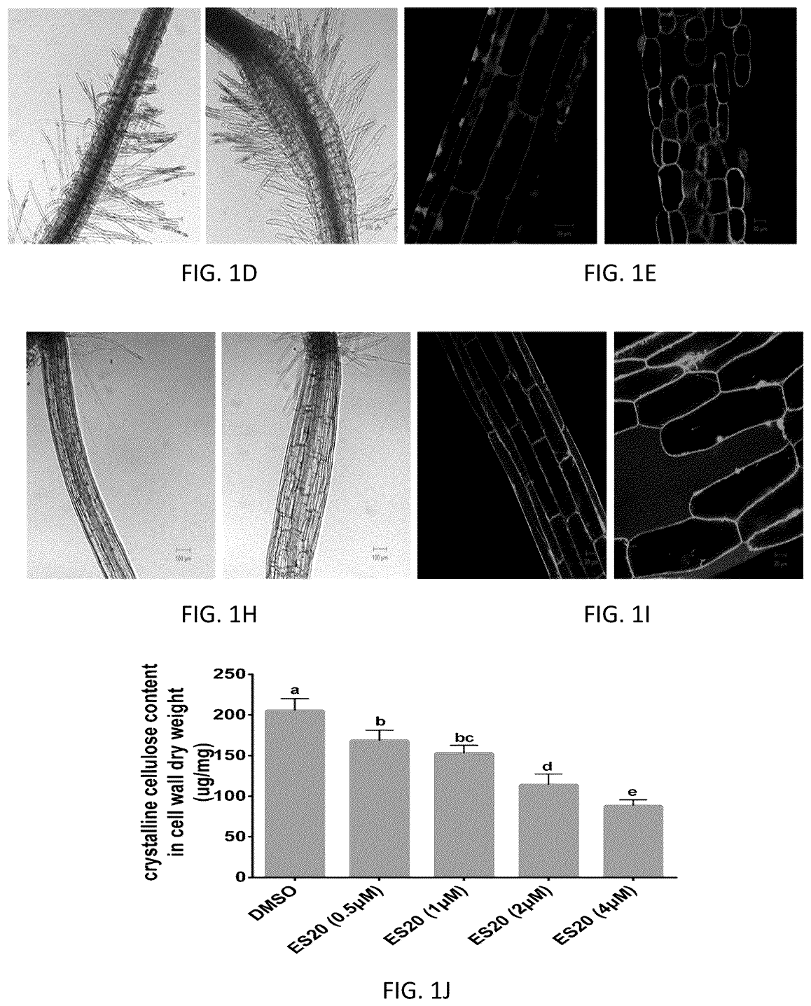

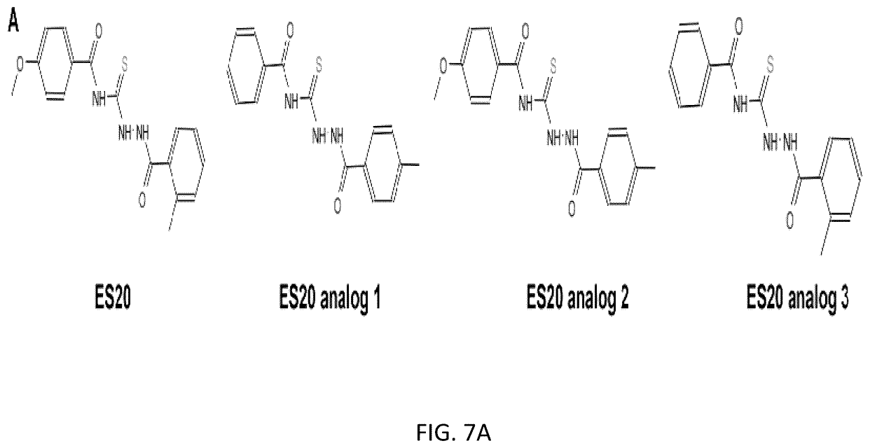

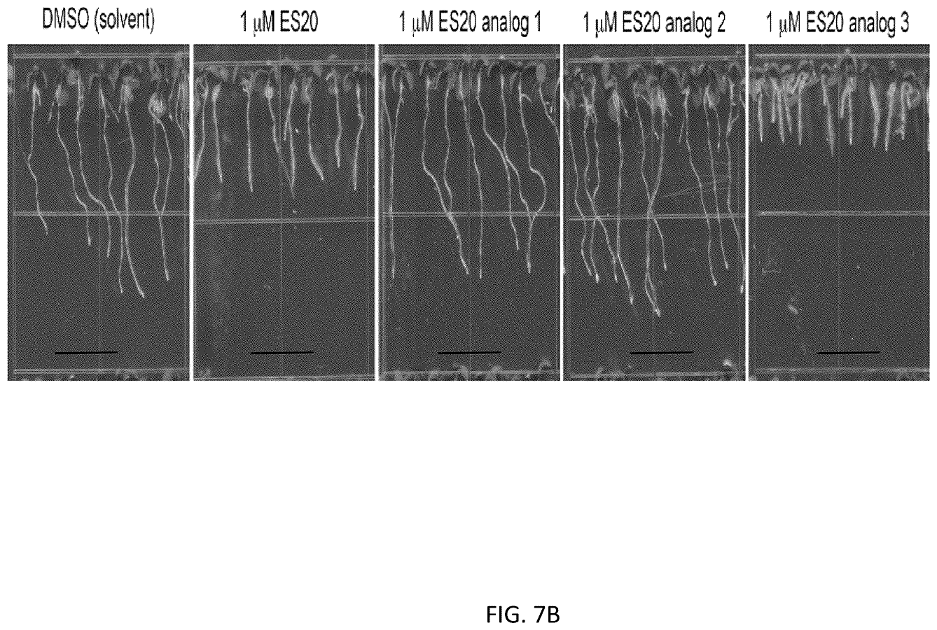

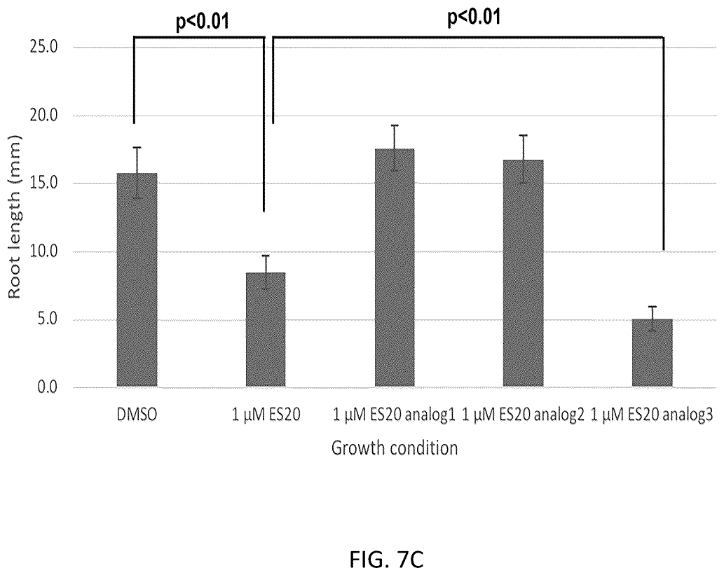

As it is disclosed herein, a cellulosin refers to a class of compounds that acts as an inhibitor toward cellulose synthase (CesA), an enzyme that catalyzes the synthesis of cellulose. Examples of a cellulosin include Endosidin20 (ES20) (FIG. 1A) and analogues 1, 2 and 3 shown in FIG. 7A.

In some embodiments, the composition of a herbicide may be formulated in various dosage forms, including, but not limited to, dry formulation, liquid formulation, granular or pellet formulation. The practice and information are known in the arts. In some other embodiments, the final product of the composition disclosed herein may be formulated as a suspension, a liquid spray, a powder, a nanoparticle, or an aerosol, together with one or more adjuvants, excipients or carriers.

In another aspect, the present invention also may include those polypeptides which exhibit at least 80%, more preferably at least 90% identity, and most preferably at least 95% identity to a polypeptide sequence selected from the group of sequences set forth above.

In some illustrative embodiments, the present invention relates to a mutant gene having the sequence of SEQ ID NOS: 3, 7, 9, 11, 13, 15, 17, 19, 21, 23, 25, 27, 29, 31, 33, 35, 37, 39, 41, 43, 45, 49, 51, and 55, or a gene of 80% or more sequence identity having said mutated gene, respectively.

In some illustrative embodiments, the present invention relates to a cell or a plant having the mutant gene disclosed herein.

In some illustrative embodiments, the present invention relates to a protein having the sequence of SEQ ID NOS: 4, 8, 10, 12, 14, 16, 18, 20, 22, 24, 26, 28, 30, 32, 34, 36, 38, 40, 42, 44, 46, 50, 52, and 56, or a polypeptide of 80% or more sequence identity having said mutated conserved amino acid residue, respectively.

In some illustrative embodiments, the present invention relates to a cell or a plant expressing the protein or polypeptide disclosed herein.

In some illustrative embodiments, the present invention relates to an isolated and cloned mutant gene encoding a cellulose synthase comprising a cellulose synthase gene with a specified nucleic acid sequence, wherein the nucleic acid sequence comprises the nucleotide sequence of SEQ ID NOS: 3, 7, 9, 11, 13, 15, 17, 19, 21, 23, 25, 27, 29, 31, 33, 35, 37, 39, 41, 43, 45, 49, 51, and 55, or a gene of 80% or more sequence identity having said mutated gene, respectively. In some other embodiments, the present invention relates to a cell or a plant having at least a copy of those genes.

In some illustrative embodiments, the present invention relates to a cell or a plant having a mutant gene, wherein said mutant gene comprises the sequence of SEQ ID NOS: 3, 7, 9, 11, 13, 15, 17, 19, 21, 23, 25, 27, 29, 31, 33, 35, 37, 39, 41, 43, 45, 49, 51, and 55, or a gene of 90% or more sequence identity having said mutated gene, respectively.

In some illustrative embodiments, the present invention relates to a cell or a plant expressing a protein, wherein said protein comprises the sequence of SEQ ID NOS: 4, 8, 10, 12, 14, 16, 18, 20, 22, 24, 26, 28, 30, 32, 34, 36, 38, 40, 42, 44, 46, 50, 52, and 56, or a polypeptide of 90% or more sequence identity having said mutated conserved amino acid residue, respectively.

In some illustrative embodiments, the present invention relates to a method for weed control of a crop comprising cloning and expressing a mutant gene having the sequence of SEQ ID NOS: 3, 7, 9, 11, 13, 15, 17, 19, 21, 23, 25, 27, 29, 31, 33, 35, 37, 39, 41, 43, 45, 49, 51, and 55, or a gene of 90% or more sequence identity having said mutated gene, respectively, in said crop, and applying an inhibitor of cellulose synthase to the field of said crop.

In some illustrative embodiments, the present invention relates to a method for weed control of a crop comprising cloning and expressing a protein having the sequence of SEQ ID NOS: 4, 8, 10, 12, 14, 16, 18, 20, 22, 24, 26, 28, 30, 32, 34, 36, 38, 40, 42, 44, 46, 50, 52, and 56, or a polypeptide of 90% or more sequence identity having said mutated conserved amino acid residue, respectively, in said crop, and applying an inhibitor of cellulose synthase to the field of said crop.

In some illustrative embodiments, the present invention relates to a compound of the following general formula (I)

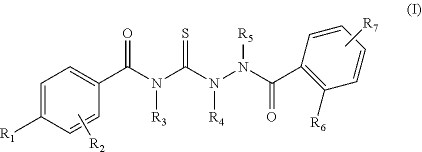

or an acceptable salt thereof, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy; and
R₃, R₄, R₅ and R₆ are independently hydrogen or an alkyl.

In some illustrative embodiments, the present invention relates to a compound of the following general formula (I), wherein the compound is

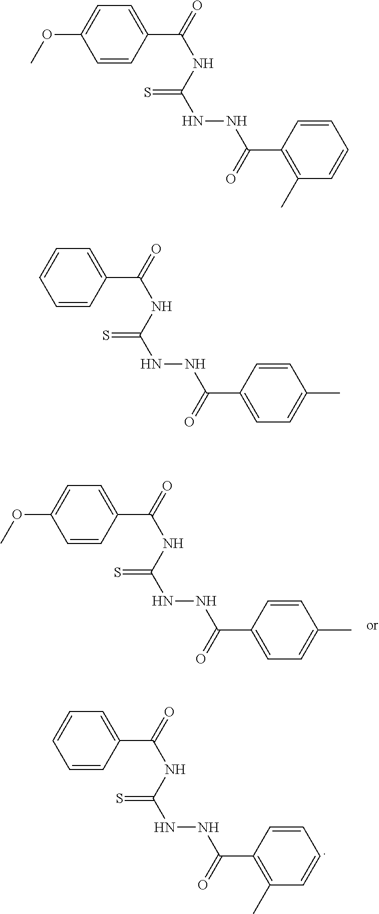

In some illustrative embodiments, the present invention relates to a cellulose synthase inhibitor having the following general formula (I)

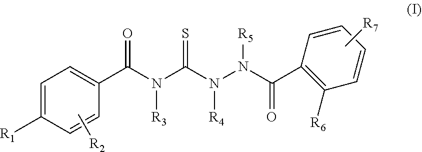

or an acceptable salt thereof, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy; and
R₃, R₄, R₅ and R₆ are independently hydrogen or an alkyl.

In some illustrative embodiments, the present invention relates to a cellulose synthase inhibitor of the following general formula (I), wherein the inhibitor is

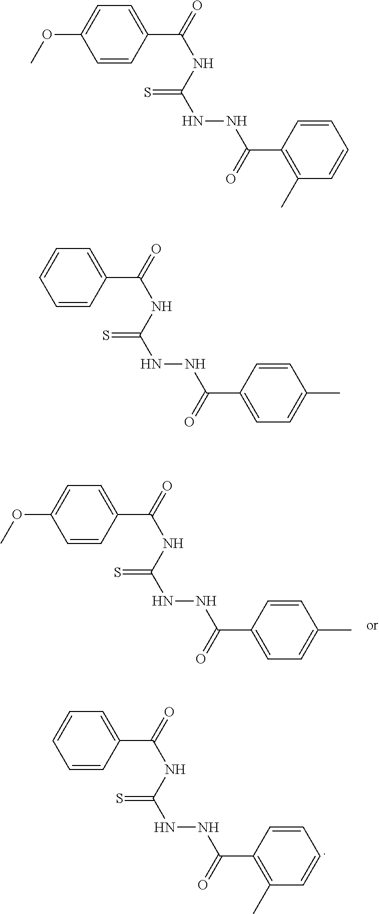

In some illustrative embodiments, the present invention relates to a herbicide having the following general formula (I)

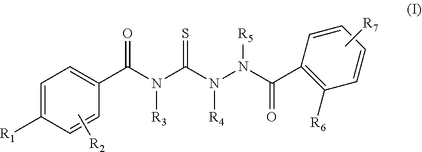

or an acceptable salt thereof, together with one or more diluents, excipients or carriers, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy;
and R₃, R₄, R₅ and R₆ are independently hydrogen or an alkyl.

In some illustrative embodiments, the present invention relates to a herbicide of the following general formula (I), wherein the herbicide is

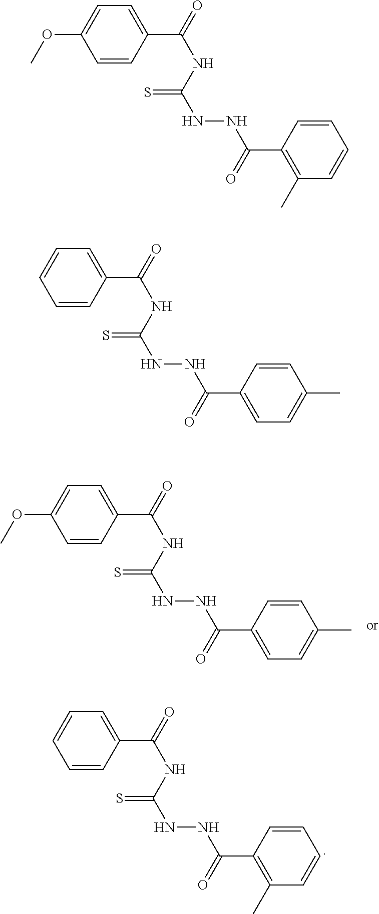

In some illustrative embodiments, the present invention relates to a herbicide of the following general formula (I), wherein the herbicide is

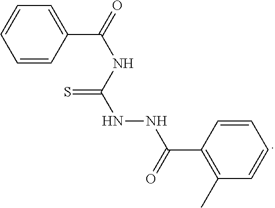

In some illustrative embodiments, the present invention relates to a method for weed control of a plant, comprising the step of applying an effective amount of an herbicide having the following general formula (I)

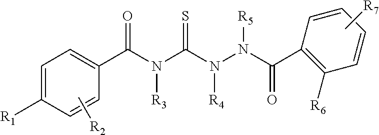

or an acceptable salt thereof, to the field of said plant, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy;
and R₃, R₄, R₅ and R₆ are independently hydrogen or an alkyl.

In some illustrative embodiments, the present invention relates to a method for weed control of a plant, comprising the step of applying an effective amount of an herbicide having the following general formula (I), wherein said herbicide is

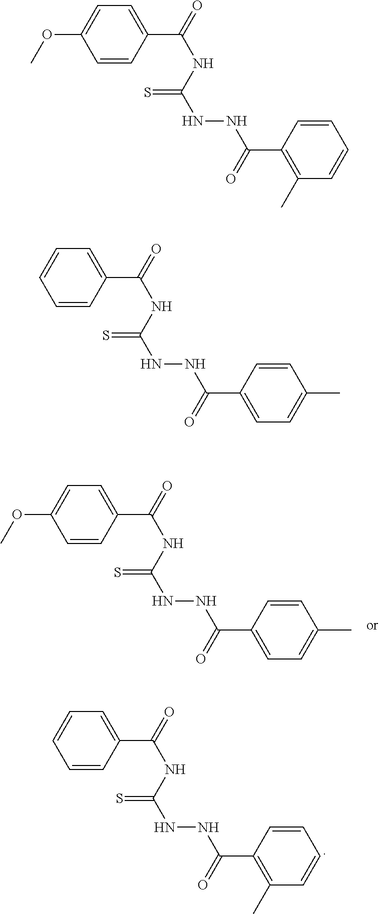

In some illustrative embodiments, the present invention relates to a method for weed control of a plant, comprising the step of applying an effective amount of an herbicide having the following general formula (I), wherein said herbicide is

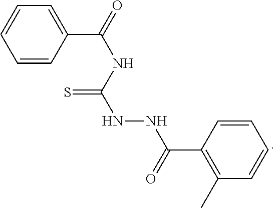

In some illustrative embodiments, the present invention relates to a method for treating a bacterial infection of a patient comprising administrating a therapeutically effective amount of a compound of formula (I), together with one or more diluents, excipients or carriers,

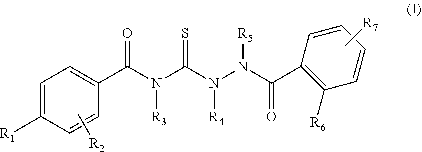

or a pharmaceutically acceptable salt thereof, to the patient in need of relief from said infection, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy; and
R₃, R₄, R₅ and R₆ are independently hydrogen or an alkyl.

In some illustrative embodiments, the present invention relates to a method for treating a bacterial infection of a patient comprising administrating a therapeutically effective amount of a compound of formula (I), together with one or more diluents, excipients or carriers, wherein the compound is

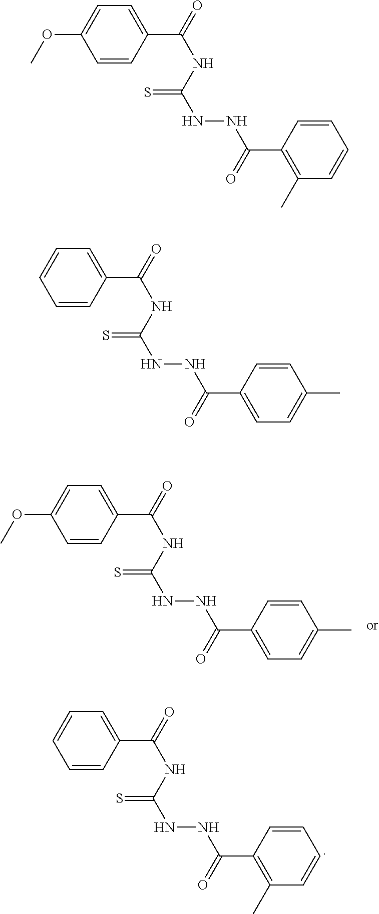

In some illustrative embodiments, the present invention relates to a method for treating a bacterial infection of a patient comprising administrating a therapeutically effective amount of a compound of formula (I), together with one or more diluents, excipients or carriers, wherein said compound is

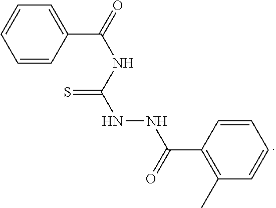 [SMILES: Cc1ccccc1C(=O)NNC(=S)NC(=O)c1ccccc1]

In some illustrative embodiments, the present invention relates to a pharmaceutical composition for treating a bacterial infection comprising a compound of formula (I), together with one or more diluents, excipients or carriers,

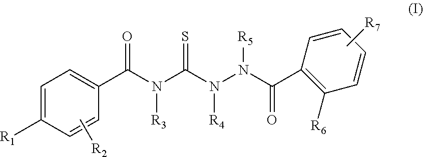

or a pharmaceutically acceptable salt thereof, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy; and
R₃, R₄, R₅ and R₆ are independently hydrogen or an alkyl.

In some illustrative embodiments, the present invention relates to a pharmaceutical composition for treating a bacterial infection comprising a compound of formula (I), together with one or more diluents, excipients or carriers, wherein the compound is

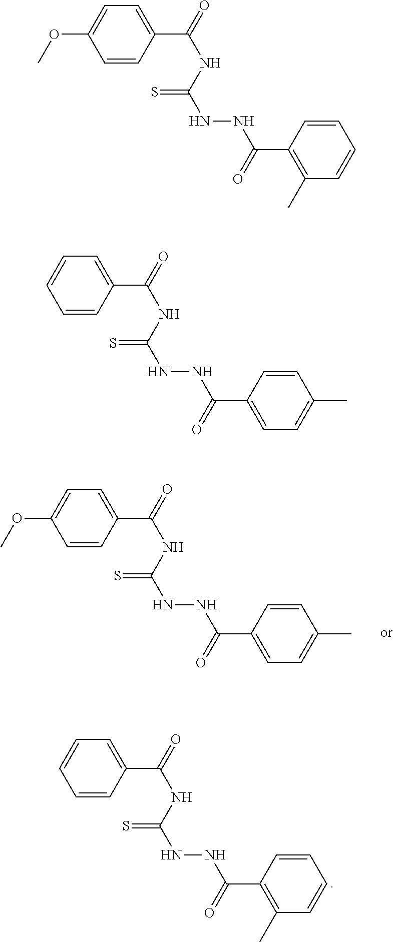

In some illustrative embodiments, the present invention relates to a pharmaceutical composition for treating a bacterial infection comprising a compound of formula (I), together with one or more diluents, excipients or carriers, wherein the compound is

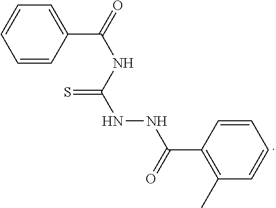 [SMILES: Cc1ccccc1C(=O)NNC(=S)NC(=O)c1ccccc1]

In some illustrative embodiments, the present invention relates to a medicament for treating a bacterial infection comprising a compound of formula (I), together with one or more diluents, excipients or carriers,

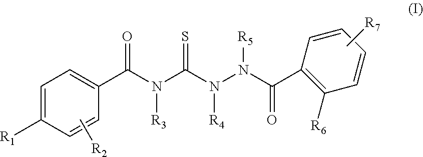

or a pharmaceutically acceptable salt thereof, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy; and
R₃, R₄, R₅ and R₆ are independently hydrogen or an alkyl.

In some illustrative embodiments, the present invention relates to a pharmaceutical composition for treating a bacterial infection comprising a compound of formula (I), together with one or more diluents, excipients or carriers, wherein the compound is

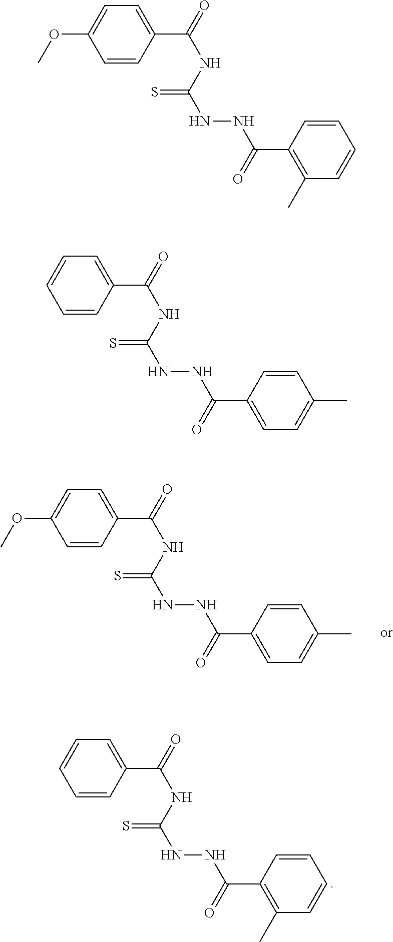

In some illustrative embodiments, the present invention relates to a pharmaceutical composition for treating a bacterial infection comprising a compound of formula (I), together with one or more diluents, excipients or carriers, wherein the compound is

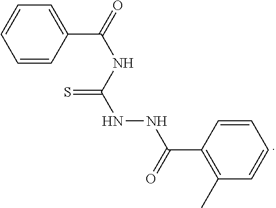 [SMILES: Cc1ccccc1C(=O)NNC(=S)NC(=O)c1ccccc1]

The following non-limiting exemplary embodiments are included herein to further illustrate the invention. These exemplary embodiments are not intended and should not be interpreted to limit the scope of the invention in any way. It is also to be understood that numerous variations of these exemplary embodiments are contemplated herein.

Like other eukaryotic organisms, plant cells contain compartmentalized organelles for controlled biochemical reactions. Membrane trafficking pathways transport molecules, such as proteins and lipids, among organelles and are essential for growth, development and environment adaptation et al. Cellulose is the polymer of β(1,4) D-glucose and is an essential cell wall component that controls plant cell survival and growth. In plants, a large portion of solar energy and CO₂ fixed through photosynthesis is used to synthesize cellulose. Although the composition of cellulose is only the linear chain of repetitive β(1,4) D-glucose units, its mechanisms of biosynthesis is poorly understood. Studies have shown that membrane trafficking pathways play essential roles in cellulose biosynthesis.

Plant cellulose is synthesized by the hexagonal rosette structured cellulose synthase complex (CSC) at the plasma membrane (Pear, J. R. et al., Proc Natl Acad Sci USA 1996, 93, 12637-12642; Arioli, T., et al., Science 1998, 279, 717-720; Giddings, T. H., J Cell Biol 1980, 84, 327-339). The exact molecular composition of CSC rosette is not clear. Each CSC is believed to contain at least 18 monomeric cellulose synthases (CesA) of three different isoforms at a 1:1:1 molar ratio (Gonneau, M. et al., Plant Physiol 2014, 166, 1709-1712; Newman, R. H. et al, Plant Physiol 2013, 163, 1558-1567). Each assembled CSC is about 25 nm in diameter with a molecular weight of at least 2 M Da. By examining cellular localization of a functional fluorescence-tagged CesA in plant cells, people have found that CSCs are not only localized to the plasma membrane, but also in the Golgi, and small CesA compartments. Plasma membrane localized CSCs co-align with the microtubules and move bidirectionally at a speed of about 300 nm/min during primary cell wall synthesis in etiolated hypocotyl epidermal cells of Arabidopsis (Paredez, A. R., Science 2006, 312, 1491-1495; Desprez, T, et al., Proc Natl Acad Sci USA 2007, 104, 15572-15577). It is understood that plasma membrane localized CSCs that move along the microtubules are performing cellulose polymerization. The conserved exocytic and endocytic trafficking pathways are responsible for the delivery and turnover of CSCs at the plasma membrane (Zhu, X. et al., Proc Natl Acad Sci USA 2018, 115. E3578-E3587; Bashline, L. et al., Plant Physiol. 2013, 163, 150-160; Bashline, L., et al., Proc. Natl Acad Sci. USA 2015, 112, 12870-12875), which indicates that membrane trafficking machineries are essential for the process of cellulose biosynthesis. However, the detailed mechanisms of CSC cellular trafficking have not been fully elucidated.

Plant CesA proteins are believed to have eight transmembrane domains and a large central cytoplasmic domain. The central cytoplasmic domain is located between two transmembrane domains are located at the N-terminal region of CesA and six transmembrane domains at the C-terminal of CesA. The central cytoplasmic domain of plant CesAs contain 492 to 536 amino acids, depending on isoforms and species (Kumar, M., et al., Phytochemistry, 2015, 112, 91-99). The central cytoplasmic domain of plant CesAs contains conserved amino acids that are responsible for the glycosyl transferase activities required for cellulose catalytic synthesis, as has been found in bacteria cellulose synthase (BcsA) (Morgan, J. L., et al., Nat Struct Mol Biol 2014, 21, 489-496; Morgan, J. L., et al., Nature 2016, 531, 329-334). Plant CesAs also contain two plant specific regions, plant conserved region (P-CR) and class specific region (CSR), in their central cytoplasmic domain. Based on structural studies from BcsA and sequence comparison between plant and bacteria cellulose synthases, it is believed that the central cytoplasmic domain of plant CesAs catalyze the synthesis of cellulose. However, it is poorly understood how the cellulose catalytic synthesis process is integrated to the trafficking of CesAs.

By screening chemical libraries using Arabidopsis thaliana, we discovered a small molecule Endosidin20 (ES20) that reduced plant growth, inhibited anisotropic cell expansion and reduced crystalline cellulose synthesis (see Table 1). In an ES20-suppression genetic screen, we discovered a number of mutant alleles of Arabidopsis CesA6 that are less sensitive to ES20. Ten of the those mutant alleles of cesa6 contain mis-sense mutations in amino acids located in the central cytoplasmic domain and one is located at the third transmembrane domain that is close to the central cytoplasmic domain. Structure modeling and molecular docking experiments indicate that ES20 could bind to a pocket in the conserved catalytic domain of CesAs. Eight of the mutated amino acids in ES20-insensitive mutants are located in the catalytic domain where ES20 is believed to bind. We used biochemical assay to show that ES20 physically interacted with the central cytoplasmic domain of CesA, where UDP-glucose, the substrate for cellulose synthesis, also binds. Based on our modeled structure and hypothesized binding sites, we created eight new transgenic lines that carrying missense mutations in amino acids that are close to the hypothesized ES20 and UDP-glucose binding site. Interestingly, five of these eight mutations lead to reduced sensitivity to ES20. We also discovered that upon short-term ES20 treatment, plasma membrane localized YFP-CesA6 is mostly removed. We show that ES20 competes with UDP-glucose, the substrate for cellulose biosynthesis, to inhibit cellulose biosynthesis and the competition reduced CesA exocytic trafficking.

ES20 is a growth inhibitor that reduces crystalline cellulose content.

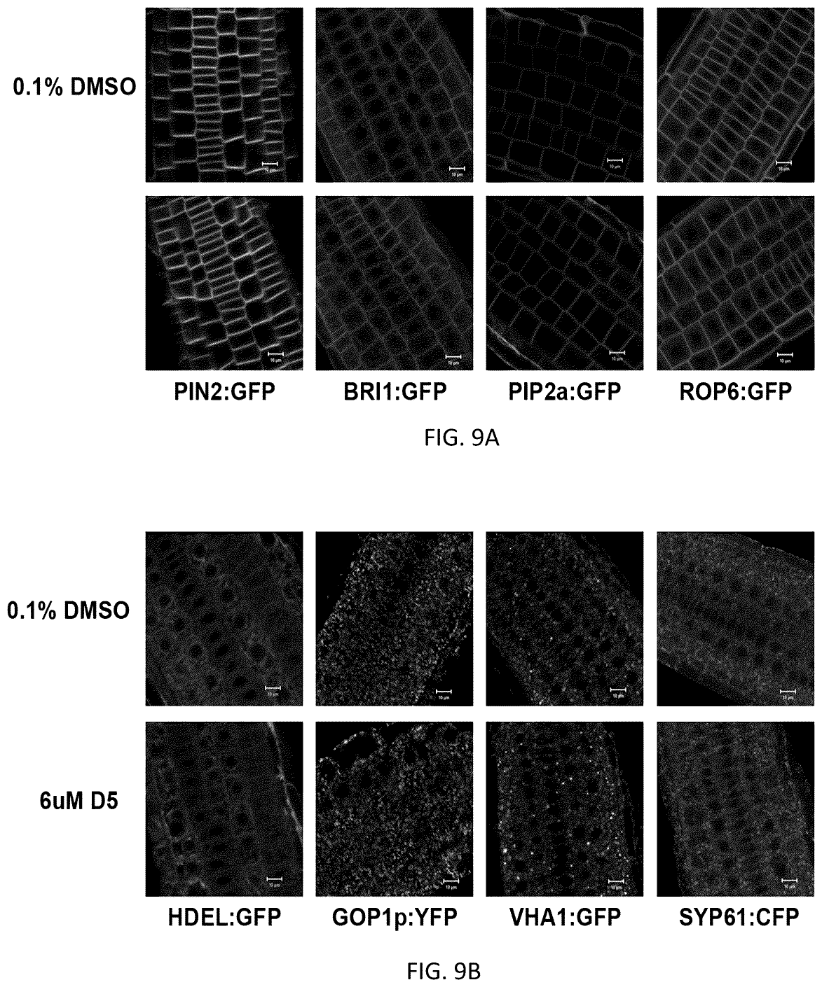

In order to identify small molecules that could be used in studying plant membrane trafficking, chemical libraries have been screened first using tobacco pollen growth assay and then Arabidopsis live cell imaging assay (Drakakaki G. et al., Proc Natl Acad Sci USA 2011, 108, 17850-17855). In primary screen, small molecules that inhibited pollen gemination or pollen tube growth in in vitro culture condition were selected. For secondary screen, selected small molecules were tested in Arabidopsis plants expressing different fluorescence-tagged cellular markers. Small molecule ES20 (FIG. 1A) inhibited tobacco pollen tube growth but surprisingly it did not significantly affect the trafficking of two typical cargo proteins, PIN2 and BRI1. Locations of general organelle markers, such as HDEL-GFP (Endoplasmic Reticulum, ER), Gop1p-YFP (Golgi), SYP61-CFP (Trans-Golgi Network, TGN) and VHA1-GFP (TGN), are unaffected as well by ES20 treatment (FIGS. 9A and 9B). These results indicate that ES20 does not inhibit general membrane trafficking in plants. However, ES20 significantly affects cell growth.

When we grew wildtype Arabidopsis seedlings on the growth media supplemented with ES20, we observed shorter and swollen roots in a ES20 dosage dependent manner (FIGS. 1B and 1C). When we grew wildtype Arabidopsis seedlings on normal growth media and then treated them with 5 μM ES20 for 12 hours in liquid growth media, the roots became swollen in the tip region, where growth occur (FIG. 1D). Staining on root epidermal cells with fluorescence dye showed that the individual cells in root elongation zone are swollen as well after 12 hours of 5 μM ES20 treatment (FIG. 1E). Arabidopsis seedlings grown on media with ES20 under dark condition have shorter and swollen hypocotyls in a ES20 dosage dependent manner (FIGS. 1F, 1G and 1H). Individual hypocotyl epidermal cells from plants grown on ES20 media are swollen as well in compare with those grown on regular media (FIG. 1I). These growth assays indicate that ES20 inhibits cell growth and causes cells to grow in an isotropic manner instead of anisotropic manner.

The composition and organization of cell wall determines plant cell shapes. Swollen cells and organs in plants are often caused by direct or indirect disruption of cell wall biosynthesis or organization. For example, swollen cells and roots have been found in cesa mutants and in plants treated with inhibitors of cellulose synthesis and microtubule organization (Arioli, T., et al., Science 1998, 279, 717-720; Burn, J. E., et al., Plant Physiol. 2002, 129, 797-807). The retarded growth and cell swollen phenotypes indicate that ES20 could affect cell growth through interfering with cell wall biosynthesis or organization. To test whether ES20 affects cell wall composition, we took the straightforward approach of analyzing crystallize cellulose content in seedlings grown in the presence of different concentrations of ES20 (Updegraff, D. M. Anal. Biochem. 1969, 32, 420-424). We found that in dark-grown Arabidopsis hypocotyls, the content of crystallize cellulose is reduced in a ES20 dosage dependent manner. The content of crystalline cellulose in seedlings grown on M ES20 was reduced to about 50% of the seedlings grown in the absence of ES20 (FIG. 1J). The reduction of crystallize cellulose in ES20 treated seedlings indicates that ES20 affects cell growth by interfering with cell wall biosynthesis.

A group of novel cesa6 alleles are less sensitive to ES20 inhibition.

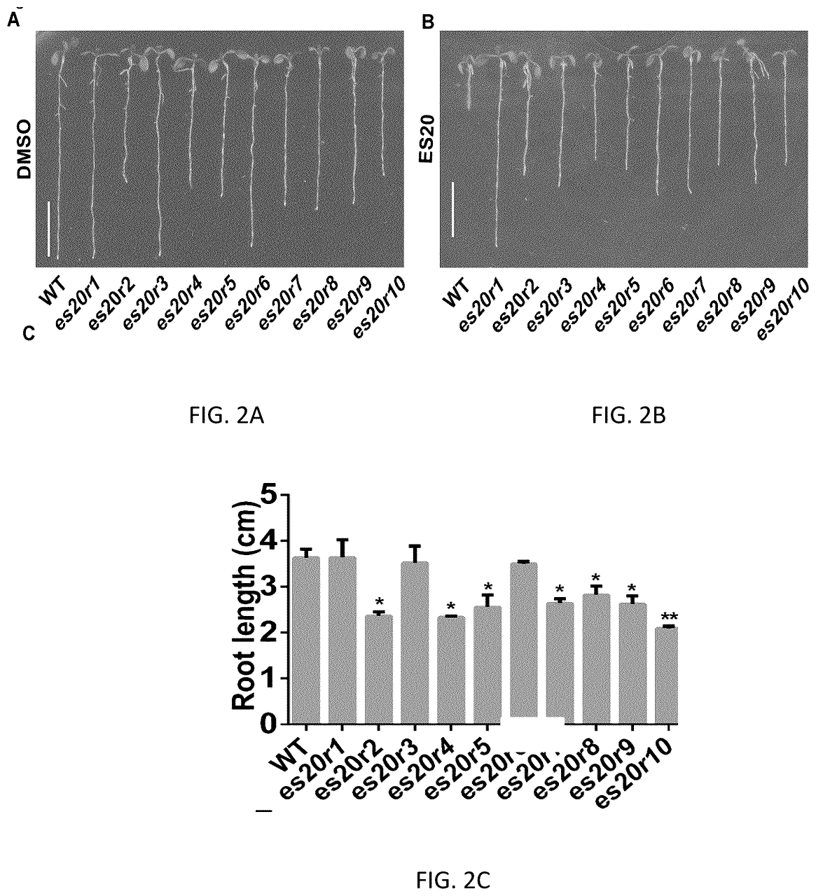

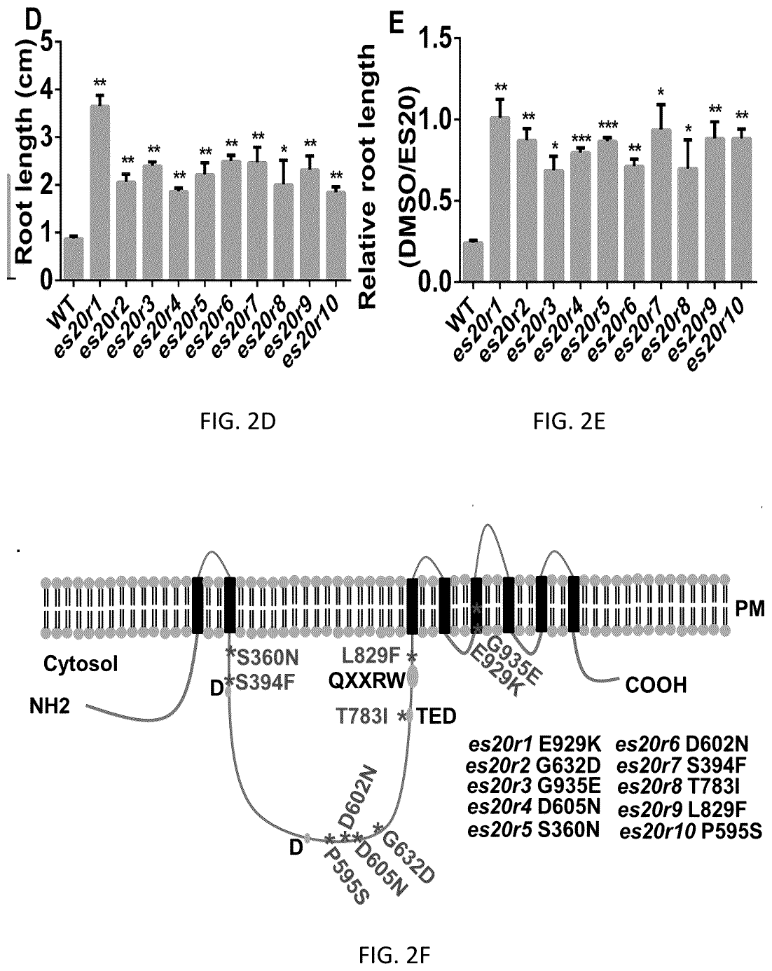

In order to identify the cell growth pathways that are targeted by ES20, we performed a chemical genetic screen for mutants that have reduced sensitivity to this growth inhibitor. About 400,000 M2 seedlings from an EMS mutagenized population in Col-0 ecotype expressing SYP61-CFP were screened for their sensitivity to 5 μM ES20, a dosage that completely arrests root growth in wild type plants. Majority of the seedlings in our screen showed chlorosis cotyledons/leaves and arrested root growths. However, we found plants that have normal green cotyledons and leaves and elongated roots. These plants became obviously heathier and stronger than other plants after two weeks of growth on ES20 and were transferred to soil to get the M3 population. After re-testing the seeds from M3, we confirmed a total of 30 individual lines that have reduced sensitivity to ES20 in growth. We generated the mapping population and used high-throughput whole genome sequencing to clone the mutant genes. After analysis on candidate SNPs in these mutants, we found that they belong to 12 mutant alleles carrying either C-T or G-A mutations. 10 out of the 12 mutants were in CesA6 and two in CesA7. After two times of backcrosses, we consistently detected less sensitivity in these mutants to ES20 (FIGS. 2A to 2E). After we further analyzed the location of the cesa6 mutations, we found that 8 of the 10 cesa6 alleles were new mutations that were located in the central cytoplasmic domain and the other two were located at the transmembrane domain at the C-terminal direction of the central cytoplasmic domain (FIG. 2F).

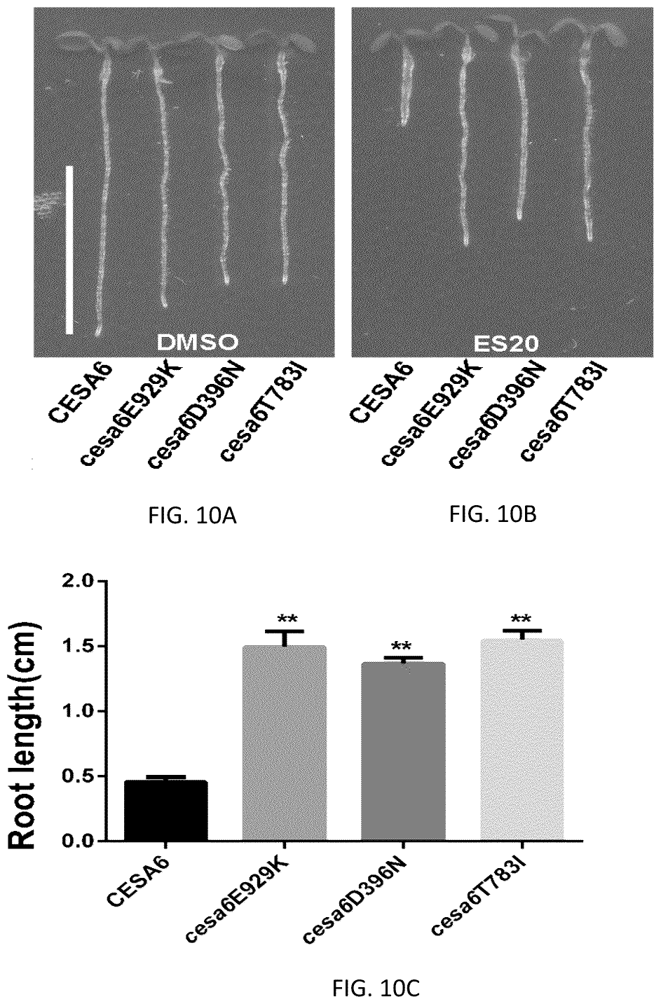

In order to confirm that the mutations at CesA6 caused the reduced sensitivity to ES20, we performed genetic complementation experiments. We cloned the genomic content of CesA6 and inserted a YFP tag to the 3′ of the coding region so that we could generate CesA6 with a N-terminal YFP fusion. We then created one of the mutations that were found in our mutants using site-directed mutagenesis. We then transformed each CesA6 construct to a loss of function cesa6 mutant named pcr1-1 (Fagard, M. et al., Plant Cell 2000, 12, 2409-2424). We found that the wild type CesA construct could rescue the growth phenotype of pcr1-1 and the mutant CesA constructs could fully or partially rescue the growth phenotype of prc1-1, depending on the mutations (FIGS. 10A, 10B, and 10C). The transgenic plants expressing wild type CesA6 construct in prc1-1 background have the same sensitivity to ES20 treatment as the wild type plants. However, the transgenic plants expressing mutant CesA6 construct in prc1-1 background have reduced sensitivity to ES20 treatment (FIGS. 10A, 10B, and 10C). These complementation experiments further confirmed that the mutations at CesA6 caused the insensitivity to ES20.

ES20 directly interacts with the central cytoplasmic domain of CesA6, likely to be at the catalytic core.

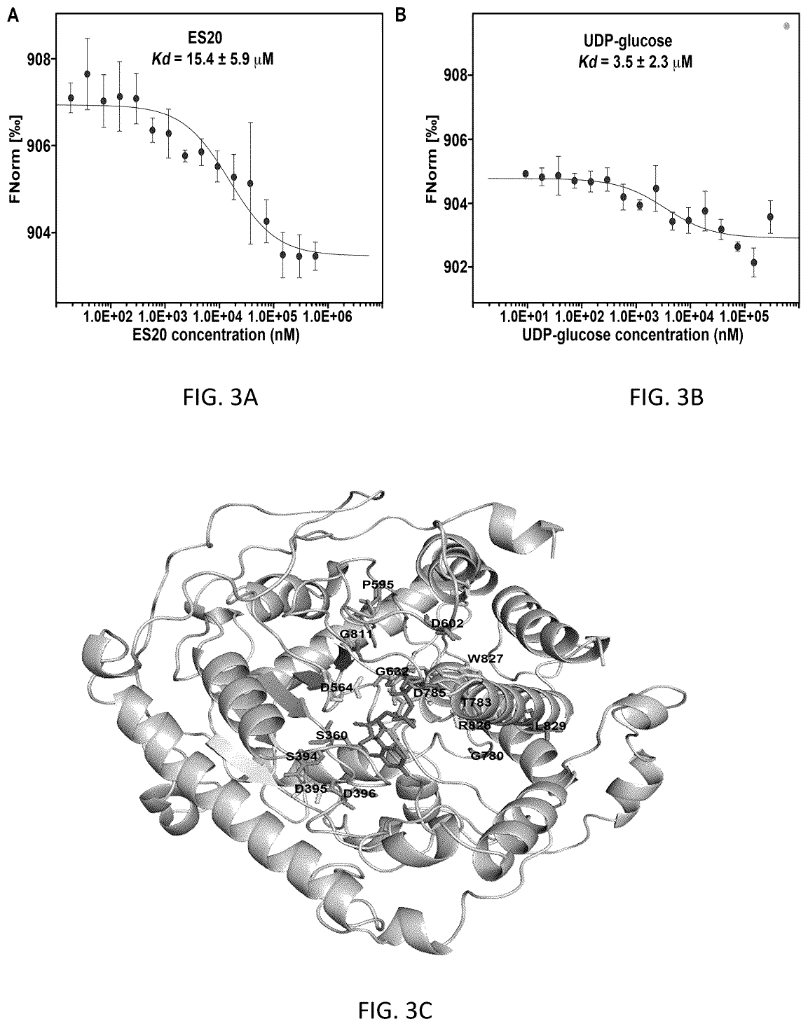

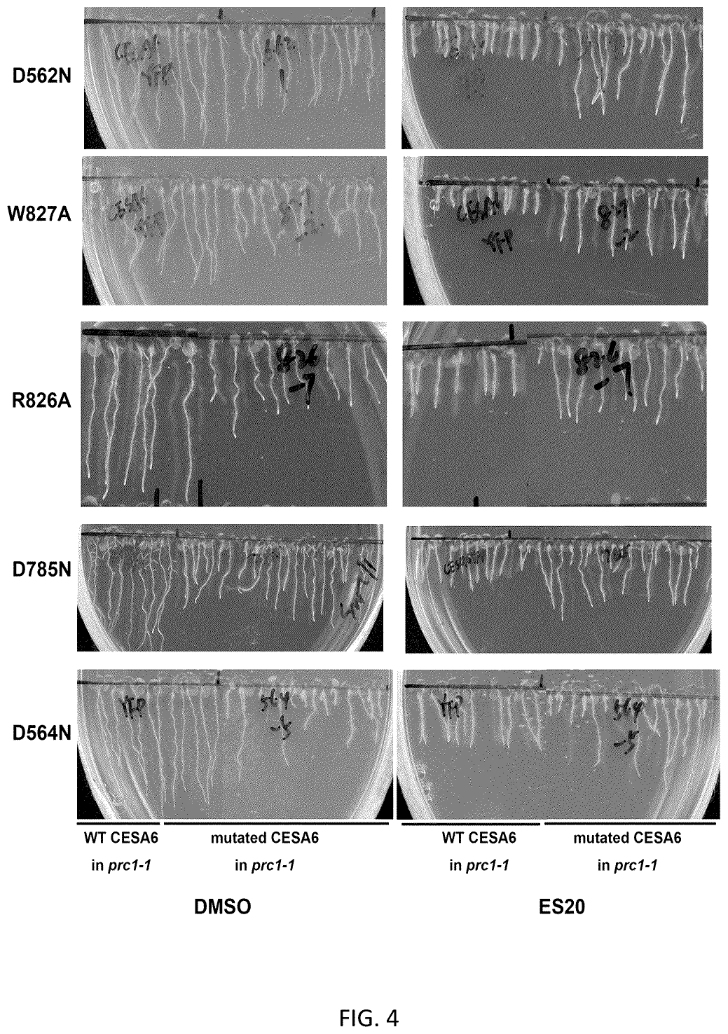

Often when a mutation occurs in the ligand binding site on a receptor protein, the ligand molecule may not be able bind to the receptor or may have reduced binding affinity to the receptor, so that the organisms could become insensitive to the ligand molecule. Multiple alleles of cesa6 that are less sensitive to ES20 made us hypothesize that ES20 could interact directly with CesA6 to inhibit its function for cellulose biosynthesis. This hypothesis also fits our observation that ES20 reduces the crystalline cellulose content in plants. In order to test for direct action between ES20 and CesA6, we expressed and purified the central cytoplasmic domain of CesA6 from E. coli. The purified central cytoplasmic domain protein contains a SUMO-His tag at the N-terminal region and a GFP tag at the C-terminal region. We then performed microscale thermophoresis (MST) assay to test for direct interaction between ES20 and the fusion protein. The MST binding curve showed that ES20 directly interacted with CesA6 with a dissociation constant (Kd) of 15.4±5.9 μM (FIG. 3A). We also tested the interaction between UDP-glucose and CesA6 central cytoplasmic domain using the same assay. We found that UDP-glucose interacts with CesA6 with a Kd of 3.5±2.3 μM (FIG. 3B).

In order to find possible binding site for ES20 on CesA6 central cytoplasmic domain, we used computational tools to model the structure of CesA6 central cytoplasmic domain using the solved structure of Rhodobacter sphaeroidesin cellulose synthase (RsBcsA) cytoplasmic domain. We then used molecular docking to explore the possible binding sites for ES20 and UDP-glucose. Surprisingly, we found both ES20 and UDP-glucose were docked to the same pocket in the center region of modeled CesA6 central cytoplasmic domain structure (FIG. 3C). When we examined the position of mutated amino acids in our reduced sensitivity mutants, we discovered that most of these amino acids are either very close to or directly located at the hypothesized binding pocket for ES20 and UDP-glucose (FIG. 3C, green colored). Two of the mutated amino acids D396 and T783, have been found to be located at the catalytic core of RsBcsA and participate in catalysis process (Morgan, J. L., et al., Nature, 2016, 531, 329-334). The bioinformatic data matched well with our genetic observation and indicated ES20 could inhibit cellulose biosynthesis by competing with UDP-glucose for the binding site on CesA6.

In order to further validate our structural modeling and molecular docking data and to further confirm that ES20 binds to the catalytic domain of CesA6, we hypothesized that if we mutated some other amino acids in the binding pocket, we should be able to reduce the binding of ES20 and the plants should have reduced ES20 sensitivity. We selected eight amino acids that are close to the hypothesized ES20 and UDP-glucose binding site and created eight CesA6 genomic constructs that each carries one missense mutation in one of these eight amino acids. We then transformed these constructs to loss of function mutant prc1-1. We obtained eight new transgenic lines that each express one of the CesA6 genomic constructs containing a missense mutation. Then these transgenic lines were tested on their sensitivity to ES20. Five out of the eight transgenic lines expressing hypothesized mutations had reduced sensitivity to ES20 (FIG. 4). In total, 24 independent mutant alleles that have mutations around the hypothesized binding pocket discovered.

ES20 inhibits exocytic trafficking of CSCs.

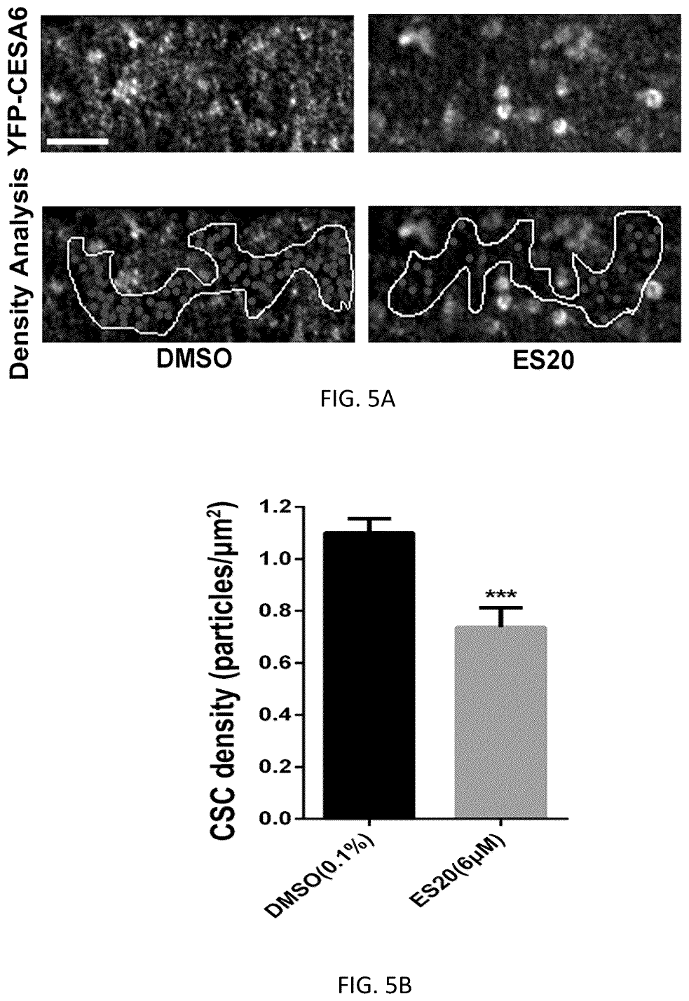

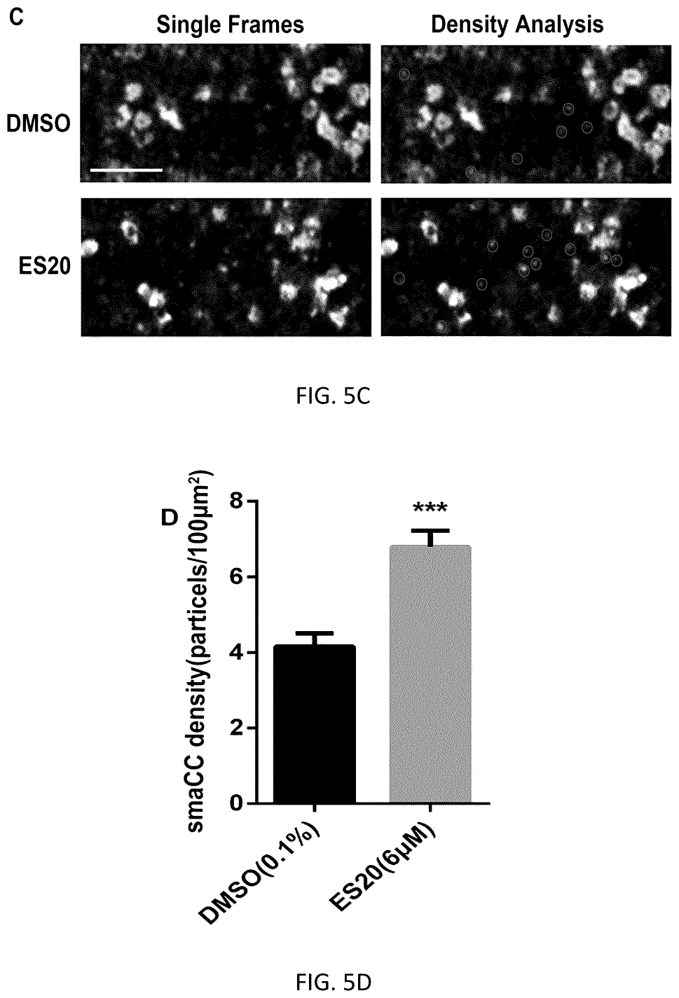

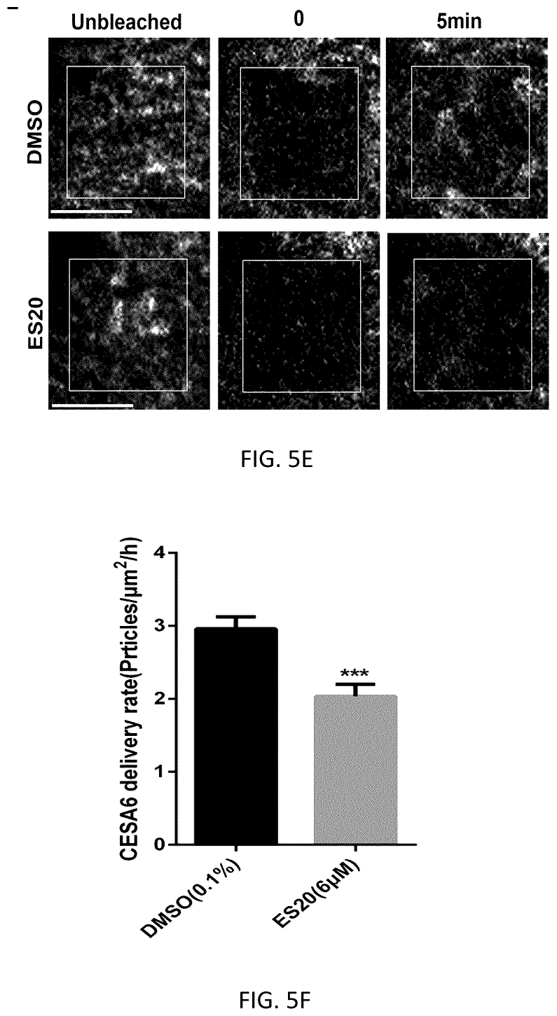

A large knowledge gap in plant biology is that it is now known how the activities of large protein complexes, such as CSC, are integrated with their cellular transport. Golgi localized CSCs are delivered to the plasma membrane to catalyze cellulose synthesis. CSCs are also localized to some small vesicles named SmaCCs. It is not known what occurs to CSCs when the catalytic core fails to bind a functional substrate. In order to understand better on the integration of CSC catalytic activity and CSC trafficking, we took a straight approach and observed the localization of CesA6 after short-term ES20 treatment. We found that after ES20 treatment, the density of plasma membrane localized CesA6 in root epidermal cells was significantly reduced (FIGS. 5A and 5B). The numbers of SmaCCs that contain CesA6 were significantly increased (FIGS. 5C and 5D). Reduced CSC density at the plasma membrane and increased SmaCCs density could be the result of increased endocytosis or decreased exocytic delivery of CSC. We then performed fluorescence recovery after photobleaching experiment to test whether the exocytic delivery of CSC was affected by ES20 treatment. We bleached YFP-CesA6 fluorescence at the plasma membrane and then observed the delivery of new CSC to the plasma membrane (FIG. 5D). We discovered that in cells treated with ES20, CSC delivery was significantly lower than the DMSO control (FIG. 5E). These results indicate that ES20 treatment affects the exocytic delivery of CSCs to the plasma membrane.

ES20 targets multiple CesA in plants.

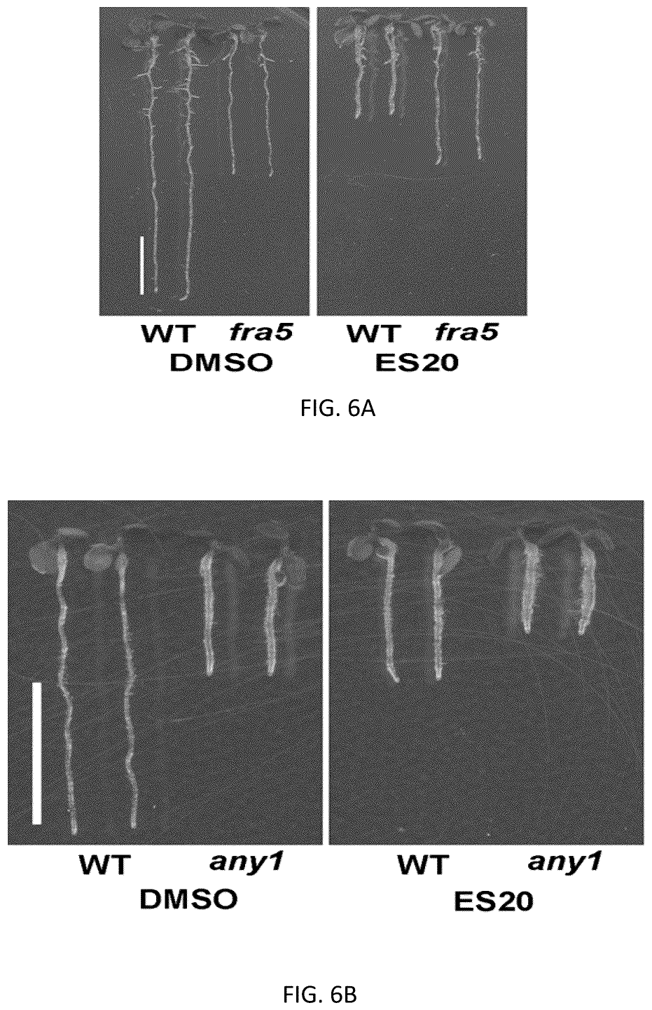

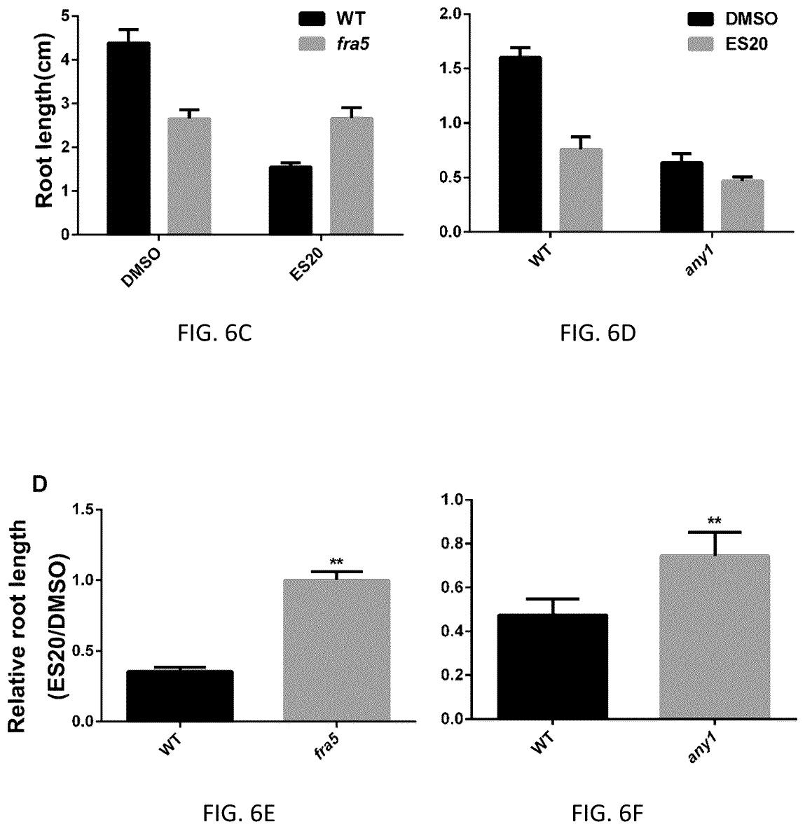

Due to the conservation of plant CesAs in their central cytoplasmic domains, we hypothesized that ES20 targeted multiple CesAs to inhibit growth. In order to test this hypothesis, we tested the response of fra5 and any1 to ES20. The mutant fra5 carries a mutation of P557T in Arabidopsis thaliana CesA7. CesA7 P557 aligns to CesA6 P595. Since the mutation of CesA6 P595S caused reduced sensitivity to ES20 in plants, we test the response of fra5 to ES20. Like CesA6 P595S mutation, fra5 plants have reduced sensitivity to ES20 too (FIGS. 6A, 6C, 6D, 6E and 6F). The mutant any1 carries a mutation of D604N in Arabidopsis CesA1. D604 in CesA1 aligns to D605 in CesA6. Since CesA6 D605N caused reduced sensitivity to ES20, we tested the response of any1 to ES20. We discovered that like CesA6 D605N, the mutation of CesA1 D604N made plants to be less sensitive to ES20 (FIGS. 6B, 6C, 6D, 6E and 6F). Reduced sensitivity to ES20 in mutants of CesA7 and CesA1 indicates that ES20 targets multiple CesAs in plants.

Analog3 of ES20 is more potent than ES20 in inhibiting growth.

In order to identify key structure in ES20 that causes its inhibition activity, we collected three close analogs of ES20 (FIG. 7A) and tested for their effect in inhibiting plant growth. We found that ES20 analog 1 and analog 2 did not inhibit Arabidopsis growth at 1 μM concentration. However, ES20 analog 3 is more potent than ES20 in inhibiting Arabidopsis growth (FIGS. 7B, and 7C).

ES20 is active in crops and weeds.

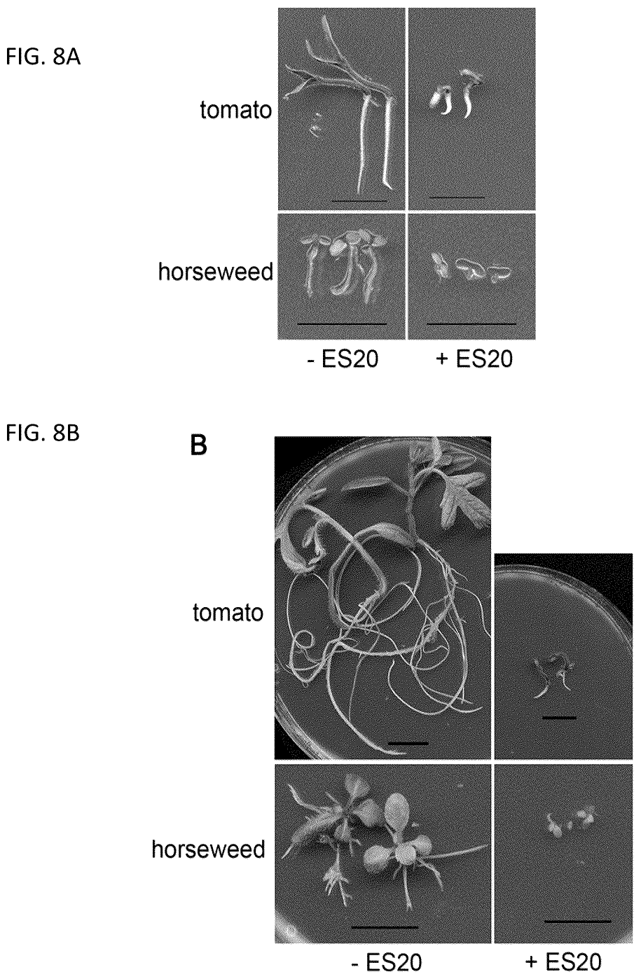

Because cellulose synthase is highly conserved in plants and ES20 targets the conserved catalytic domain, we hypothesize that ES20 is active in different plants, including common weeds. We tested this hypothesis by growing tomato and horseweed on growth media containing ES20. We found that ES20 could significantly inhibit the growth of both tomato and horseweed (FIG. 8). This indicates that ES20, and probably its more potent analogs, can serve as herbicide for weed control.

Use of cellulose synthase inhibitor ES20 or an analogue thereof for treatment of bacterial infections, especially for those bacteria that produce and form a biofilm at the site of infection.

Cellulose is also a component of the extracellular matrix of biofilm that is composed of groups of microorganism cells, including bacteria (Ziemba, C., et al., NPJ Biofilms Microbiomes 2016, 2, 1; Limoli, D. H., et al, Microbiol Spectr. 2015, 3; Mann, E. E., et al., FEMS Microbiol Rev. 2012, 36, 893-916). Biofilms cause human and animal tooth decay and many other types of infectious diseases such as urinary tract infection, cystic fibrosis and middle-ear infections (Bjarnsholt, T. APMIS 2013, Suppl., 1-51). The extracellular matrix of biofilms protects them from biotic and abiotic stresses and made biofilms difficult to manipulate. The application of general antibiotics often fails to cure biofilm infections and there is great need to develop alternative methods to control biofilms (Rybtke, M., et al., J. Mol. Biol. 2015, 427, 3628-3645). Inhibition of cellulose synthesis has the potential to inhibit the formation of biofilms extracellular matrix and makes it possible to overcome biofilm antibiotic resistance and control corresponding diseases caused by biofilms more efficiently.

Strategies for producing crops that are resistant to ES20.

We have discovered that single amino acid change in model plant Arabidopsis thaliana is sufficient to create plants that are resistant to ES20. A total of 24 amino acid mutations that lead to resistance to ES20 are disclosed in this present application. Those mutats are found in AtCesA4, AtCesA6, AtCesA7 and AtCesA8. These amino acids are highly conserved among different plant species. The same mutation in these conserved amino acids of any crops will have resistance to a cellulose synthase inhibitor, such as ES20. Creating a cell or a crop plant by incorporating those mutated genes and conveying cellulose synthase inhibitor resistance to the cell or a crop plant are known in the art. Furthermore, one can create a crop plant that is resistant to a cellulose synthase inhibitor, including ES20 and its analogues, using three different ways. (1). Clone the genomic construct of CesA6 homolog in a crop and create a single nucleotide mutation in the genomic construct. Then transform the construct that carries the single nucleotide mutation to the crop so that the construct expresses a CesA6 homolog protein carrying a mutated amino acid. The expression of CesA6 homologs with the mutated amino acid will allow the crop plant to be resistant to a cellulose synthase inhibitor. (2). Create a mutation in CesA6 homolog construct in a crop and express the construct under an inducible promoter. The mutated CesA6 homolog will only be expressed when a cellulose synthase inhibitor, like ES20, is applied. (3). Use CRISPR-cas9 to edit a crop genome to create a mutation at the amino acid that we have disclosed herein. For specific examples, see D. K. Sohoo, et al., Plant Biotechnology Journal (2013) 11, pp. 362-372.; and J. L. Acebes, et al., Cellulose Biosynthesis Inhibitors. In: Cellulose: Structure and Properties . . . Editors: A. Lejeune, T. Deprez, pp. 39-73. Nova Science Publishers, Inc. 2010. ISBN: 978-1-60876-388-7. General Methods: NMR spectra were recorded on Bruker spectrometers (¹H at 500 MHz; ¹³C at 125 MHz.

TABLE 1
Wild Type and Mutant Genes and Protein Products Listing

| Gene | | | Location | Location | | | Protein |
|---|---|---|---|---|---|---|---|
| SEQ ID | Mutant | | on genomic | on coding | Codon | Amino acid | SEQ ID |
| NO: | gene name | Gene | DNA | sequence | change(s) | change(s) | NO: |
| 1 | wild type | CESA4 | | | | | 2 |
| 3 | cesa4^{C53F} | CESA4 | 267G-T | 158G-T | TGC-TTC | C53→F | 4 |
| 5 | wild type | CESA6 | | | | | 6 |
| 7 | cesa6^{D396N} | CESA6 | 1916G-A | 1186G-A | GAT-AAT | D396→N | 8 |
| 9 | cesa6^{D564N} | CESA6 | 2804G-A | 1690G-A | GAT-AAT | D564→N | 10 |
| 11 | cesa6^{D562N} | CESA6 | 2798G-A | 1684G-A | GAT-AAT | D562→N | 12 |
| 13 | cesa6^{D602N} | CESA6 | 2918G-A | 1804G-A | GAT-AAT | D602→N | 14 |
| 15 | cesa6^{D605N} | CESA6 | 2927G-A | 1813G-A | GAT-AAT | D605→N | 16 |
| 17 | cesa6^{D785N} | CESA6 | 3717G-A | 2353G-A | GAT-AAT | D785→N | 18 |
| 19 | cesa6^{E929K} | CESA6 | 4309G-A | 2785G-A | GAA-AAA | E929→K | 20 |
| 21 | cesa6^{G632D} | CESA6 | 3092G-A | 1895G-A | GGT-GAT | G632→D | 22 |
| 23 | cesa6^{G780S} | CESA6 | 3702G-A | 2338G-A | GGT-AGT | G780→S | 24 |
| 25 | cesa6^{G811E} | CESA6 | 3796G-A | 2432G-A | GGA-GAA | G811→E | 26 |
| 27 | cesa6^{G935E} | CESA6 | 4328G-A | 2804G-A | GGA-GAA | G935→E | 28 |
| 29 | cesa6^{L829F} | CESA6 | 3849C-T | 2485C-T | CTT-TTT | L829→F | 30 |
| 31 | cesa6^{L286F} | CESA6 | 1503C-T | 856C-T | CTT-TTT | L286→F | 32 |
| 33 | cesa6^{P595S} | CESA6 | 2897C-T | 1783C-T | CCT-TCT | P595→S | 34 |
| 35 | cesa6^{Q823E} | CESA6 | 3831C-G | 2467C-G | CAA-GAA | Q823→E | 36 |
| 37 | cesa6^{S360N} | CESA6 | 1809G-A | 1079G-A | AGT-AAT | S360→N | 38 |
| 39 | cesa6^{S394F} | CESA6 | 2400C-T | 1181C-T | TCT-TTT | S394→F | 40 |
| 41 | cesa6^{R826A} | CESA6 | 3840/3841 | 2476/2477 | CGA-GCA | R826→A | 42 |
| | | | CG-GC | CG-GC | | | |
| 43 | cesa6^{W827A} | CESA6 | 3843/3844 | 2479/2480 | TGG-GCG | W827→A | 44 |
| | | | TG-GC | TG-GC | | | |
| 45 | cesa6^{T783I} | CESA6 | 3712C-T | 2348C-T | ACC-ATC | T783→I | 46 |
| 47 | wild type | CESA7 | | | | | 48 |
| 49 | cesa7^{A243V} | CESA7 | 1607C-T | 728C-T | GCT-GTT | A243→V | 50 |
| 51 | cesa7^{D100N} | CESA7 | 829G-A | 298G-A | GAC-AAC | D100→N | 52 |
| 53 | wild type | CESA8 | | | | | 54 |
| 55 | cesa8^{P174S} | CESA8 | 943C-T | 520C-T | CCT-TCT | P174→S | 56 |

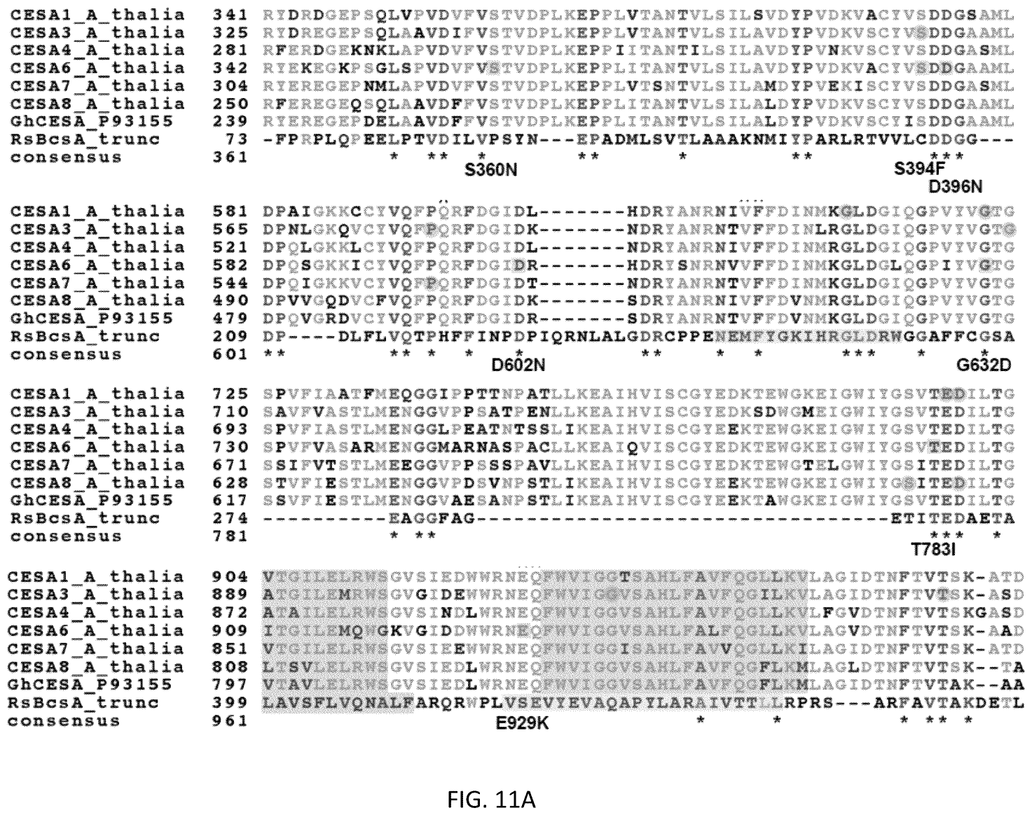

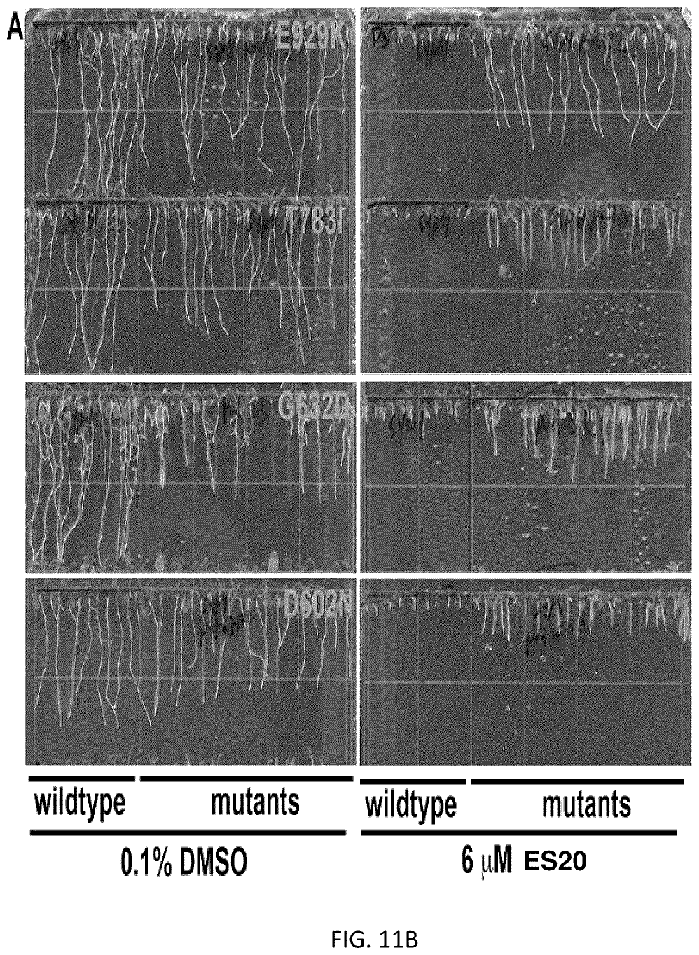

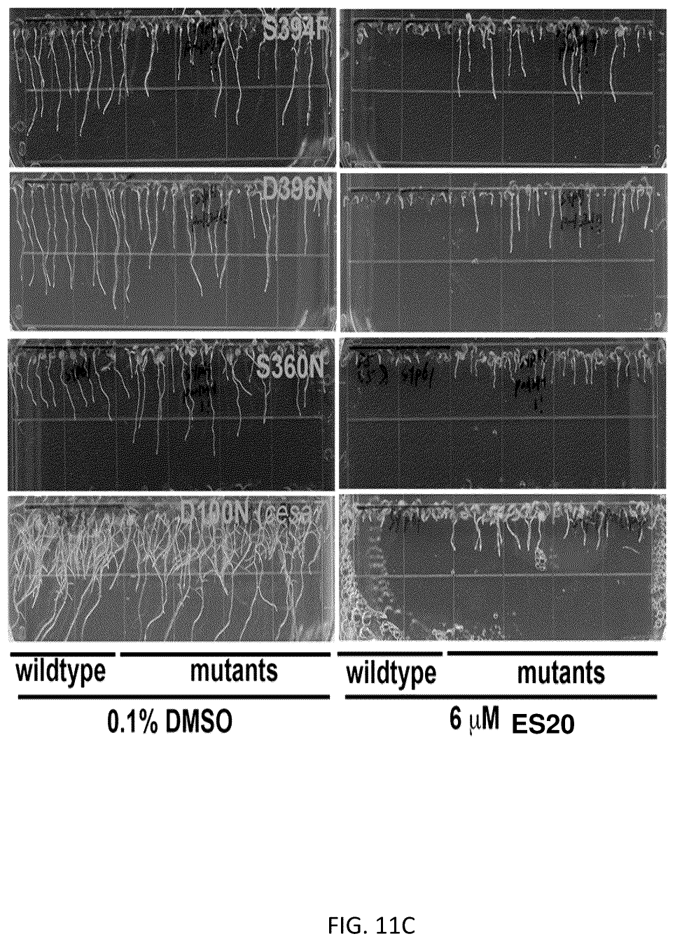

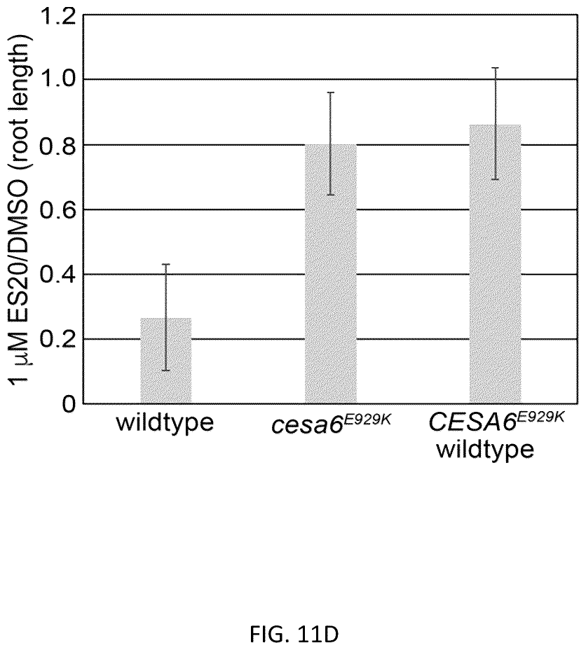

The mutations we found are located in Arabidopsis thaliana CesA6 (AT5G64740), CesA4 (AT5G44030), CesA7 (AT5G17420) and CesA8 (AT4G18780) genes. Single nucleotide change in these mutants causes single amino acid change in CesA proteins and lead to reduced sensitivity to cellulosin (endosidin20 or ES20). The mutations we have discovered are listed in Table 1 and FIG. 11A showing the conserved amino acid residues of those subtypes of cellulose synthases. Representative resistant phenotypes are shown in FIG. 11B. We also found that when we express CesA6 constructs that carry the mutations in wildtype Arabidopsis, the plants have reduced sensitivity to Cellulosin (FIG. 11C). This indicates that we can create Cellulosin resistant plants in two ways. One is to create mutations in CesA6 homology gene in crop. The second is to express the mutant CesA6 in crops without knocking out endogenous CesA genes.

### Synthesis of Compound ES20

Chemical shifts (δ) were given in ppm with reference to solvent signals [¹H NMR: CDCl₃ (7.26); ¹³C NMR: CDCl₃ (77.2)]. Column chromatography was performed on silica gel. All reactions were conducted under argon atmosphere and all solvents and reagents were used as obtained from commercial sources without further purification (Scheme 1).

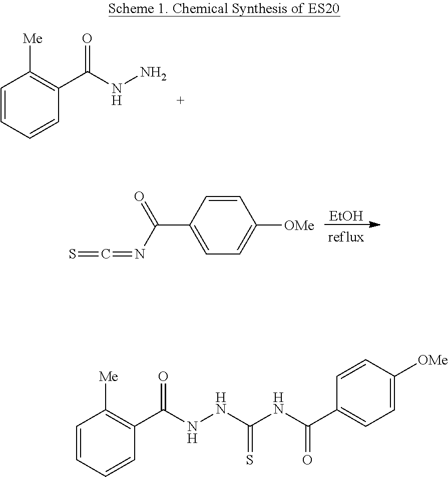 [SMILES: "EtOH" ; Cc1ccccc1C(=O)NN ; COc1ccc(C(=O)N=C=S)cc1 ; COc1ccc(C(=O)NC(=S)NNC(=O)c2ccccc2C)cc1]

o-Toluic hydrazide (1.50 g, 10.0 mmol) was added to a stirred solution of ethanol (40 mL) followed by 4-methoxybenzoyl isothiocyanate (1.93 g, 10.0 mmol) at room temperature. The solution was heated up to reflux under argon for 15 mins. Ethanol was removed under vacuum to give the crude product as a yellow solid. The crude product was recrystallized in ethanol to give 2.00 g purified product as a white crystal in 59% yield.

HRMS (ESI) [M+H⁺] calculated for C₁₇H₁₇N₃O₃S: 344.1063, found: 344.1064; FTIR (neat, cm⁻¹) vₘₐₓ 3216, 1667, 1603, 1525, 1498, 1428, 1258, 1174, 1028, 842, 758; ¹H NMR (500 MHz, CDCl₃) δ: 9.48 (s, 1H), 8.95 (s, 1H), 7.87 (d, J=8.8 Hz, 2H), 7.60 (d, J=7.6 Hz, 1H), 7.42 (t, J=7.5 Hz, 1H), 7.30 (d, J=7.8 Hz, 2H), 7.01 (d, J=8.9 Hz, 2H), 3.90 (s, 3H), 2.56 (s, 3H); ¹³C NMR (126 MHz, CDCl₃) δ: 171.6, 166.0, 164.4, 164.1, 137.9, 131.6, 131.4, 129.8, 127.6, 126.0, 123.0, 114.5, 55.7, 20.2.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It should be understood by those skilled in the art that various alternatives to the embodiments described herein may be employed in practicing the claims without departing from the spirit and scope as defined in the following claims.

What is claimed is:

1. A mutant gene having the sequence of SEQ ID NOS: 3, 7, 9, 11, 13, 15, 17, 19, 21, 23, 25, 27, 29, 31, 33, 35, 37, 39, 41, 43, 45, 49, 51, and 55, or a gene of 80% or more sequence identity having said mutated gene, respectively, wherein said gene confers resistance to a cellulose synthase inhibitor.

2. A cell or a plant having the mutant gene of claim 1.

3. The mutant gene according to claim 1, wherein said gene expresses a protein having the sequence of SEQ ID NOS: 4, 8, 10, 12, 14, 16, 18, 20, 22, 24, 26, 28, 30, 32, 34, 36, 38, 40, 42, 44, 46, 50, 52, and 56, or a polypeptide of 80% or more sequence identity having said mutated conserved amino acid residue, respectively, wherein said protein confers resistance to a cellulose synthase inhibitor.

4. (canceled)

5. An isolated and cloned mutant gene encoding a cellulose synthase comprising a cellulose synthase gene with a specified nucleic acid sequence, wherein the nucleic acid sequence comprises the nucleotide sequence of SEQ ID NOS: 3, 7, 9, 11, 13, 15, 17, 19, 21, 23, 25, 27, 29, 31, 33, 35, 37, 39, 41, 43, 45, 49, 51, and 55, or a gene of 80% or more sequence identity having said mutated gene, respectively.

6. A cell or a plant having the gene of claim 5.

7. (canceled)

8. The cell or a plant according to claim 6, wherein said mutant gene expresses a protein having the sequence of SEQ ID NOS: 4, 8, 10, 12, 14, 16, 18, 20, 22, 24, 26, 28, 30, 32, 34, 36, 38, 40, 42, 44, 46, 50, 52, and 56, or a polypeptide of 90% or more sequence identity having said mutated conserved amino acid residue, respectively.

9. A method for weed control of a crop comprising cloning and expressing a mutant gene having the sequence of SEQ ID NOS: 3, 7, 9, 11, 13, 15, 17, 19, 21, 23, 25, 27, 29, 31, 33, 35, 37, 39, 41, 43, 45, 49, 51, and 55, or a gene of 90% or more sequence identity having said mutated gene, respectively, in said crop, and applying an inhibitor of cellulose synthase to the field of said crop.

10. The method for weed control of a crop according to claim 9, wherein said mutant gene expresses a protein having the sequence of SEQ ID NOS: 4, 8, 10, 12, 14, 16, 18, 20, 22, 24, 26, 28, 30, 32, 34, 36, 38, 40, 42, 44, 46, 50, 52, and 56, or a polypeptide of 90% or more sequence identity having said mutated conserved amino acid residue, respectively, in said crop, and applying an inhibitor of cellulose synthase to the field of said crop.

11. The method for weed control of a crop according to claim 9, wherein said inhibitor of cellulose synthase is a compound of the following general formula: 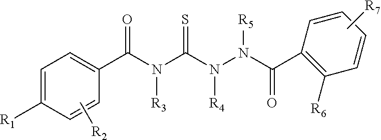
or an acceptable salt thereof, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy; and
R₃, R₄, R₅ and R₆ are independently hydrogen or an alkyl.

12. The method for weed control of a crop according to claim 11, wherein the compound is

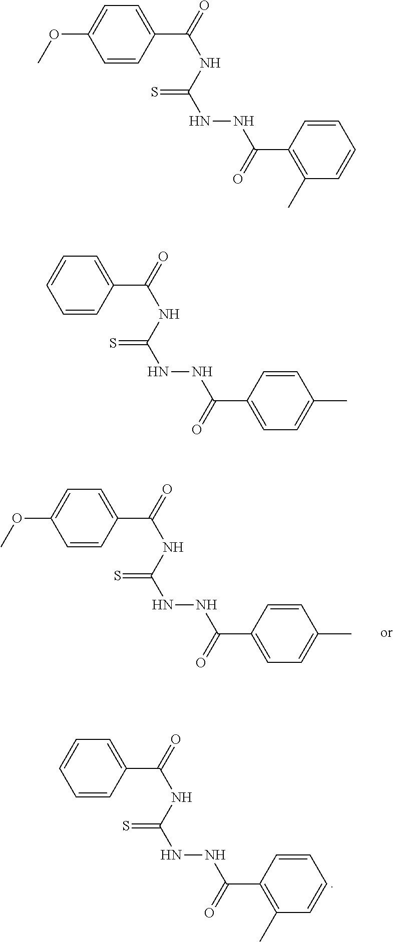

13. A cellulose synthase inhibitor having the following general formula (I) 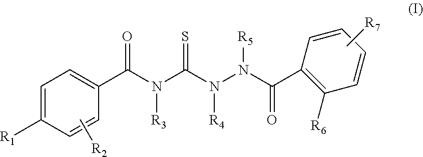
or an acceptable salt thereof, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy; and
R₃, R₄, R₅ and R₆ are independently hydrogen or an alkyl.

14. The cellulose synthase inhibitor of claim 13, wherein the inhibitor is

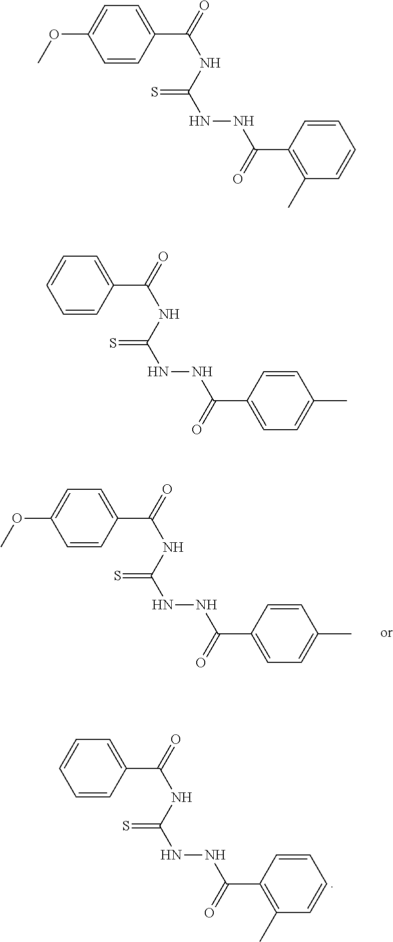

15. The cellulose synthase inhibitor of claim 13, wherein said inhibitor is a herbicide.

16. (canceled)

17. (canceled)

18. A method for weed control of a plant, comprising the step of applying an effective amount of an herbicide having the following general formula (I) 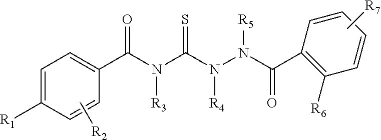
or an acceptable salt thereof, to the field of said plant, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy;
and R₃, R₄, R₅ and R₆ are independently hydrogen or an alkyl.

19. The method of claim 18, wherein said herbicide is

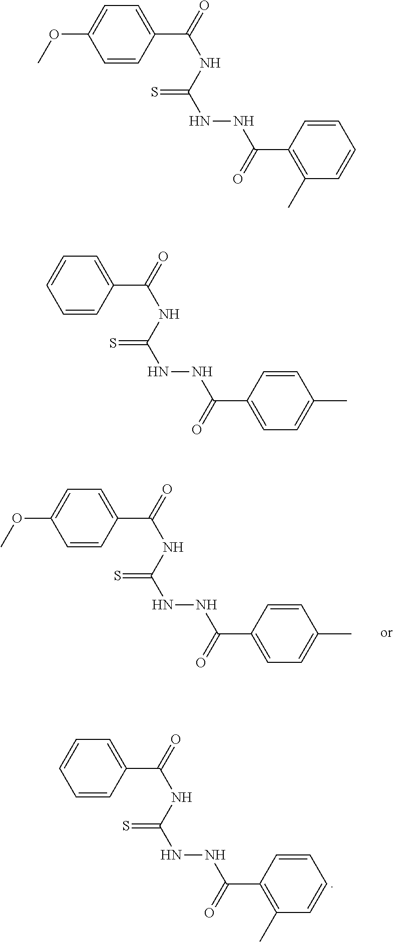

20. (canceled)

21. A method for treating a bacterial infection of a patient comprising administrating a therapeutically effective amount of a compound of formula (I), together with one or more diluents, excipients or carriers, 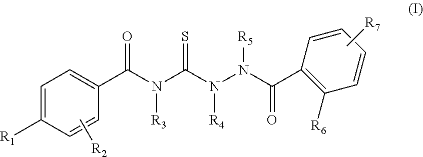
or a pharmaceutically acceptable salt thereof, to the patient in need of relief from said infection, wherein
R₁ is hydrogen, a halo, an alkyl, alkenyl, or alkyloxy;
R₂ and R₇ represent four individual substituents independently selected from the group comprising hydrogen, a halo, an alkyl, alkenyl, and alkyloxy; and
R₃, R₄, R₅ and R₆ are independently hydrogen or an alkyl.

22. The method of claim 21, wherein the compound is

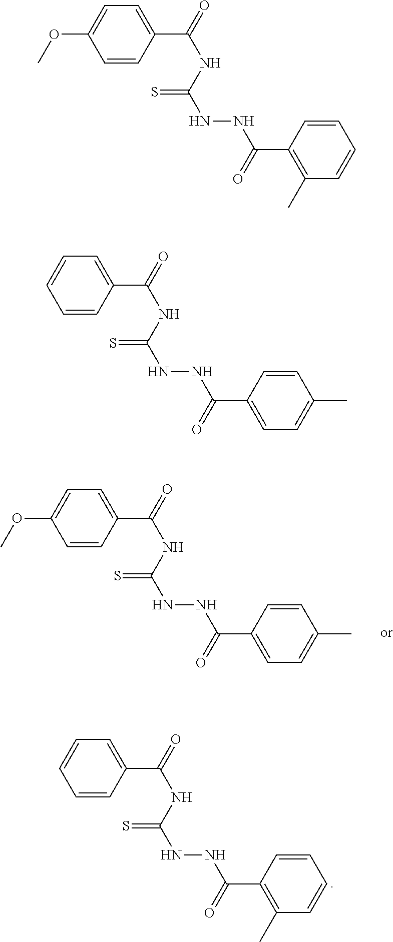

23.-29. (canceled)
